(19) United States
(12) Patent Application Publication
(10) Pub. No.: US 2021/0291009 A1
(43) Pub. Date: Sep. 23, 2021

(54) INTELLIGENT EXERCISE MATS, ACCESSORIES, ASSEMBLIES, SYSTEMS, AND METHODS OF USING THE SAME

(71) Applicants: Kevin Paganini, New York, NY (US); Carolina Salgado, New York, NY (US)

(72) Inventors: Kevin Paganini, New York, NY (US); Carolina Salgado, New York, NY (US)

(21) Appl. No.: 17/207,682

(22) Filed: Mar. 21, 2021

Related U.S. Application Data
(60) Provisional application No. 62/992,988, filed on Mar. 22, 2020.

(51) Int. Cl. A63B 21/00; A63B 71/06; A63B 24/00

(52) U.S. Cl. CPC A63B 21/4037; A63B 71/0622; A63B 2225/50; A63B 2071/0625; A63B 2220/806; A63B 24/0003

(57) ABSTRACT

The present disclosure is related to improved exercise mats, as well as related assemblies, systems, apparatuses, and methods of use. The exercise mats can be integrated with various electronic accessories. Some or all of the electronic accessories can be powered by one or more power sources incorporated into the exercise mats. In some cases, the exercise mats can communicate with an online platform and/or mobile application that provides various functionality to enhance user experiences with the exercise mats.

### CROSS-REFERENCE TO RELATED APPLICATIONS

This application claims the benefit of, and priority to, U.S. Provisional Patent Application No. 62/992,988 filed on Mar. 22, 2020, which is herein incorporated by reference in its entirety.

### TECHNICAL FIELD

This disclosure is related to intelligent exercise mats that include integrated electronic accessories. This disclosure also describes various systems and methods for fabricating, utilizing, and implementing the intelligent exercise mats and integrated accessories.

### BACKGROUND

Exercise mats, such as yoga mats and other fitness mats, are commonly utilized for various activities, such as yoga, Pilates, meditation, calisthenics, weight training, and/or other related activities. In many cases, the exercise mats provide a soft cushion for users' body parts (e.g., feet, tailbone, knees, etc.) when performing these activities, which avoids pain that may be incurred from performing these activities on hard floor surfaces. In addition to providing comfort, other benefits of exercise mats include improving users' balance, stability, and grip when performing activities.

Many individuals often desire to use various accessories to compliment their works out or other activities. In one example, users may desire devices that are capable of providing aromatherapy to supplement activities such as yoga, Pilates, and meditation. In another example, users may desire to use audio or radio devices to play music during weight training and calisthenics activities. In a further example, users commonly utilize wired phone chargers to charge their mobile devices (e.g., mobile smart phones) when using the exercise mats.

Individuals who desire to use these and other accessories while utilizing their exercise mats are forced not only to purchase the desired accessories, but also to transport the accessories with the exercise mats to desired locations (e.g., yoga studios, homes, outdoor locations, and/or other desired location) where the activities will be performed. Additionally, in many cases, the individuals can only utilize certain accessories if a power outlet is nearby to power the accessories. Furthermore, when users desire to adjust the settings of the accessories (e.g., change a song a radio or audio device, adjust the output of an oil diffuser, turn on/off, etc.), the individuals must stop the activities they are performing on the exercise mats and walk to the location of the accessories, thereby interrupting their work out, meditation, and/or other activity they are engaged in.

### BRIEF DESCRIPTION OF THE DRAWINGS

To facilitate further description of the embodiments, the following drawings are provided in which:

FIG. 1A illustrates an exemplary exercise mat according to certain embodiments;

FIG. 1B illustrates an exemplary exercise mat according to certain embodiments;

FIG. 1C illustrates an exemplary exercise mat according to certain embodiments;

FIG. 2 illustrates exemplary accessories that may be integrated with or connected to an exercise mat according to certain embodiments;

FIG. 3A illustrates an exemplary exercise mat having an accessory dashboard according to certain embodiments;

FIG. 3B illustrates an exemplary exercise mat having an accessory dashboard according to certain embodiments;

FIG. 3C illustrates an accessory dashboard incorporated into various standalone structures according to certain embodiments;

FIG. 3D illustrates an exemplary exercise mat having an accessory dashboard according to certain embodiments;

FIG. 3E illustrates an exemplary exercise mat having an accessory dashboard according to certain embodiments;

FIG. 3F illustrates a top surface of an upper housing portion of an accessory dashboard according certain embodiments;

FIG. 3G illustrates a bottom surface of an upper housing portion of an accessory dashboard according certain embodiments;

FIG. 3H illustrates a top surface of a lower housing portion of an accessory dashboard according certain embodiments;

FIG. 3I illustrates a bottom surface of a lower housing portion of an accessory dashboard according certain embodiments;

FIG. 3J illustrates a lower housing portion of an accessory dashboard connected to an exercise mat according certain embodiments;

FIG. 3K is a front view of an accessory dashboard according to certain embodiments;

FIG. 3L is a top view of an accessory dashboard according to certain embodiments;

FIG. 3M is a side view of an accessory dashboard according to certain embodiments;

FIG. 3N is a perspective view of an accessory dashboard according to certain embodiments;

FIG. 4 illustrates an exemplary system according to certain embodiments;

FIG. 5 illustrates an exemplary online platform according to certain embodiments;

FIG. 6A is a block diagram demonstrating exemplary components included in ultrasonic or humidifying diffuser according to certain embodiments;

FIG. 6B is a block diagram demonstrating exemplary components included in a nebulizing or atomizing diffuser according to certain embodiments;

FIG. 6C is a block diagram demonstrating exemplary components included in evaporative diffuser according to certain embodiments;

FIG. 6D is a block diagram demonstrating exemplary components included in electric or heat diffuser according to certain embodiments;

FIG. 7A is an exploded view of an ultrasonic diffuser according to certain embodiments;

FIG. 7B is a perspective view of an ultrasonic diffuser according to certain embodiments; and

FIG. 7C is perspective view of ultrasonic diffuser according to certain embodiments.

### DETAILED DESCRIPTION OF EXEMPLARY EMBODIMENTS

The present disclosure is related to improved exercise mats, as well as related assemblies, systems, apparatuses, and methods of use. Exercise mats can be improved by integrating various electronic accessories, devices, and features directly or indirectly into the exercise mats, thereby enhancing the functionality of the exercise mats and improving experiences of exercise mat users. For example, the exercise mats can be equipped with electronic accessories that enable the exercise mats to dispense fragrances and aromas (e.g., for use in aromatherapy routines), output audio (e.g., music and/or exercise routines), display images and/or videos (e.g., video exercise and/or meditation routines), illuminate an area in the vicinity of the exercise mats (e.g., using lighting components), track or monitor user movements on the mats (e.g., using sensors embedded into the exercise mats), monitor health information of users (e.g., using heartbeat sensors and/or other vital monitoring sensors), record workout routines (e.g., using microphone and/or camera devices), and/or other functions described herein. Additionally, some or all of the electronic exercise mat accessories can be powered by one or more power sources incorporated into the exercise mats.

The exercise mats can be equipped with any combination of the accessories described herein. In certain embodiments, the exercise mats can be outfitted or equipped with electronic accessories that are selected to enhance user activities related to yoga, meditation, or the like. As mentioned above, many users commonly enjoy aromatherapy and music while performing such activities. Thus, in certain embodiments, an exercise mat can be equipped with one or more electronic aroma diffusers (e.g., ultrasonic oil diffusers, atomizing oil diffusers, and/or electric diffusers) that are configured to emit mists including essential oils in the vicinity of the exercise mat, as well as one or more audio devices (e.g., speakers) that can output audio (e.g., music and/or exercise routine instructions) while the exercise mat is being used. Additionally, the exercise mat may include one or more communication modules (e.g., Bluetooth modules) that enables users to stream music to the audio devices on the exercise mat (e.g., using a mobile device), one or more wireless charger devices (e.g., for wirelessly charging users' mobile devices), and one or more input ports (e.g., USB ports) that enable additional accessories (e.g., fans, display screens, computers, etc.) to be connected to, and powered by, the exercise mat. All of these and other electronic accessories can be connected to, and powered by, one or more batteries integrated into the exercise mat.

The dimensions, sizes, and weights of many traditional electronic devices (e.g., such as aroma diffusers, speakers, etc.) can impede the devices from being incorporated into exercise mats, which tend to be relatively thin (e.g., 4-7 millimeters (mm) in some cases), in a practical manner. For example, attempting to connect a typical speaker and/or aroma diffuser to a top surface of an exercise mat would result in a cumbersome exercise mat configuration in which the electronic devices would protrude a significant height (e.g., several inches) above the top surface, and the weight of the exercise mat would increase significantly (e.g., double or triple in weight). In addition to having an unattractive appearance, this cumbersome configuration would not be practical to transport, would prevent the exercise mats from being rolled or folded into space-saving configurations, and would impede usage of the exercise mat itself.

The techniques described herein enable the accessories to be incorporated into the exercise mats in a manner that does not interfere with usage of the exercise mats and allows the exercise mats to be easily converted to space-saving configurations (e.g., rolled up). Various techniques are described which can be utilized to minimize the dimensions, sizes, and weights of the electronic accessories, as well as exercise mats that are integrated with the accessories.

In certain embodiments, subcomponents of the electronic accessories can be separately integrated into body portions of the exercise mats and situated adjacent to one another in a horizontal plane to minimize the height and/or depth dimensions of both the exercise mats and electronic accessories. In some cases, holes or recesses may be cut into the exercise mats and the electronic accessories can be integrated into the holes or recesses. In some cases, some or all of the accessories (or portions thereof) may be integrated between top surfaces and bottom surfaces of the exercise mats such that they are hidden from view. Additionally, or alternatively, some or all of the accessories can be incorporated into a dashboard located on the periphery of the exercise mats in locations that do not impede usage of the exercise mats. Additionally, or alternatively, some or all of the accessories can be accessible on top surfaces of the exercise mats, but the upper portions of the accessories may not protrude above the top surfaces of the mats (or may only protrude slightly above the top surfaces) to avoid interference with usage of the exercise mats. The aforementioned techniques can be combined in any manner to minimize the physical structures of the exercise mats and corresponding accessories.

In certain embodiments, the manner in which the accessories are incorporated into the exercise mats enables the mats to be rolled up or folded in space-saving configurations, and to be easily cleaned and sanitized without damaging the accessories. Additionally, in certain embodiments, one or more of the accessories included in the exercise mats can be configured to be detachable from the exercise mats (e.g., to permit cleaning and/or refilling of the accessories, minimize the weight or size of the exercise mats, and/or convert the exercise mat to a traditional exercise mat).

The exercise mats also can include one or more communication interfaces (e.g., transceivers and/or hardwire connections) that enable the exercise mats to communicate with one or more online platforms and/or computer devices over a network. For example, the exercise mats can collect various data pertaining to usage of the exercise mats (e.g., accessories used, exercise routines performed, etc.) and/or the individuals who utilize the exercise mats (e.g., user vitals and health information), and this information can be transmitted to the online platform for storage and/or analysis. The online platform also can enable the users to access pre-recorded exercise routines and/or participate in live exercise classes using one-way and/or bi-directional videoconference sessions. The online platforms accessed over the network can provide other types of content and functions as described in further detail below.

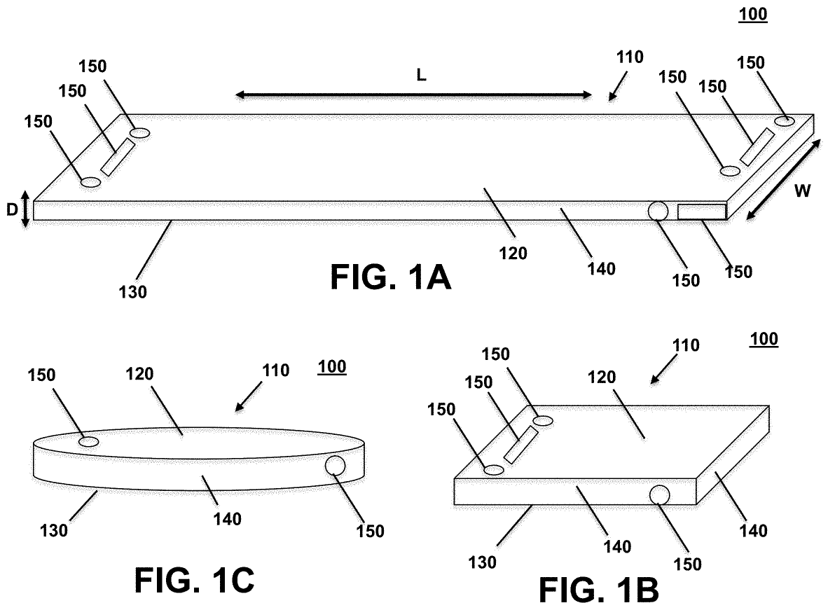

FIGS. 1A-1C disclose exemplary exercise mats 100 according to certain embodiments. The exercises mats 100 include a body portion 110 that can be constructed, fabricated, and/or formed of any material. For example, the body portions 110 of the exercise mats 100 may be constructed of one or more of the following materials: vinyl, polyvinyl chloride (PVC) foam, latex, ethylene-vinyl acetate (EVA) foam, thermoplastics, rubber, jute, cotton, cork, polyurethane foams, and/or polyurethane leathers. In certain embodiments, the materials incorporated into the body portions 110 of the exercise mats 100 can be selected from eco-friendly materials and/or anti-microbial materials.

The exercises mats 100 can be configured in any shape. For example, in some cases, the exercise mats 100 can be configured in a rectangular shape. The exercise mats 100 can additionally, or alternatively, be configured in a square, circle, oval, triangle, pentagon, hexagon, cross, and/or other shapes. FIG. 1A discloses an exemplary exercise mat 100 arranged in a rectangle configuration. FIG. 1B discloses an exemplary exercise mat 100 arranged in a square configuration. FIG. 1C discloses an exemplary exercise mat 100 arranged in a circular configuration. Other configurations of the mat 100 are also within the scope of this disclosure.

The lengths, widths, and thicknesses of the exercise mats 100 can vary. In certain embodiments (e.g., including embodiments in which the mat may be rectangular shaped), the thickness or depth (labeled “D” in FIG. 1A) of the exercise mat 100 can be in a range of 0.01 inch to 2 inches. For example, the thickness of the mats may be 1/16 inch, ⅛ inch, ¼ inch, ⅜ inch, ½ inch, 1 inch, 1.5 inches, and/or 2 inches. In certain embodiments (e.g., including certain embodiments in which the mat is rectangular shaped), the width (labeled “W” in FIG. 1A) of the exercise mats 100 can be in a range of 12 inches to 48 inches. For example, the width of the exercise mats 100 can be 20 inches, 24 inches, 30 inches, and/or 36 inches. In certain embodiments (e.g., including certain embodiments in which the mat is rectangular shaped), the length of the (labeled “L” in FIG. 1A) exercise mats 100 can be in a range of 40 inches to 100 inches. For example, the length of the exercise mats 100 can be 50 inches, 60 inches, 68 inches, 70 inches, 72 inches, 74 inches, 76 inches, and/or 80 inches.

In certain embodiments, the body portion 110 of the exercise mat 100 can include a top surface 120, a bottom surface 130, and one or more side surfaces 140 (e.g., four side surfaces in rectangular-shaped exercise mats or one continuous side surface in circular-shaped exercise mats). The top surface 120 may be used by an individual for various activities (e.g., yoga, mediation, Pilates, exercising, etc.), the bottom surface 130 may rest on an underlying ground, floor, or surface, and the side surfaces 140 can be situated between the top surfaces 120 and bottom and surfaces 140. In certain embodiments, a bottom surface 130 of the body portion 110 can include movement-resisting or grip-enabling features (e.g., textured surfaces, and/or sticky materials) to prevent the exercise mats 100 from sliding or moving during usage. The top surface 120 of the exercise mats 100 may include similar movement-resisting or grip-enabling features to prevent individuals from sliding or slipping during usage of the exercise mats 100. Some or all of the exercise mat surfaces can be fabricated with, or otherwise include, moisture-wicking materials and/or anti-microbial materials.

In certain embodiments, the exercise mats 100 may include mats that can be used for yoga, Pilates, meditation, and/or similar activities. The exercise mats 100 additionally, or alternatively, can be used for weight training, calisthenics, stretching, and/or other exercise-related activities. In certain embodiments, the exercise mats 100 can be used for non-exercise related activities (e.g., such as lounging, watching television, sleeping, camping, etc.)

One or more accessories 150 may be integrated into body portions 110 and/or other portions of the exercise mats 100 to supplement and enhance various functionalities provided by the exercise mats 100. The accessories 150 may include various devices that enable the exercise mats 100 to dispense fragrances and aromas, display images and/or videos, illuminate the exercise mat and/or adjacent areas, track movements on the mats, monitor health information of users, record workout routines, charge electronic devices, and/or perform other functions described herein. The particular accessories 150, as well as the number of accessories 150, incorporated into the exercise mats 100 can vary, and the exercise mats 100 can include any combination of the accessories 150 describes herein. For example, in some embodiments, an exercise mat 100 may include one of the accessories 150 described herein. In other embodiments, an exercise mat 150 may include multiple accessories 150 described herein.

The accessories 150 can be integrated into any surface (including the top, bottom, and/or side surfaces) of the exercise mats 100. The locations of the accessories 150 may vary. In certain embodiments, some or all of the accessories 150 may be integrated into a top surface of the exercise mats, which can be advantageous because users can easily access and/or manipulate the accessories while using the exercise mats 100. Additionally, in some cases, the accessories 150 can be located along the edge or periphery of the top surface (and/or the other surfaces), which can be advantageous because the locations of the accessories do not impede usage of the exercise mats 100. For example, the accessories 150 may be located in one or all of the corners on the top surface and/or bottom surface. In some cases, the accessories can also be located in or around the center of the exercise mats 100 and/or in central portions of the exercise mats 100. Additionally, or alternatively, the accessories 150 can be located between the top surfaces 120 and bottom surfaces 130 of the exercise mats 100 (e.g., such that they are not visible), which again can be useful for avoiding interference with using the exercise mats 100.

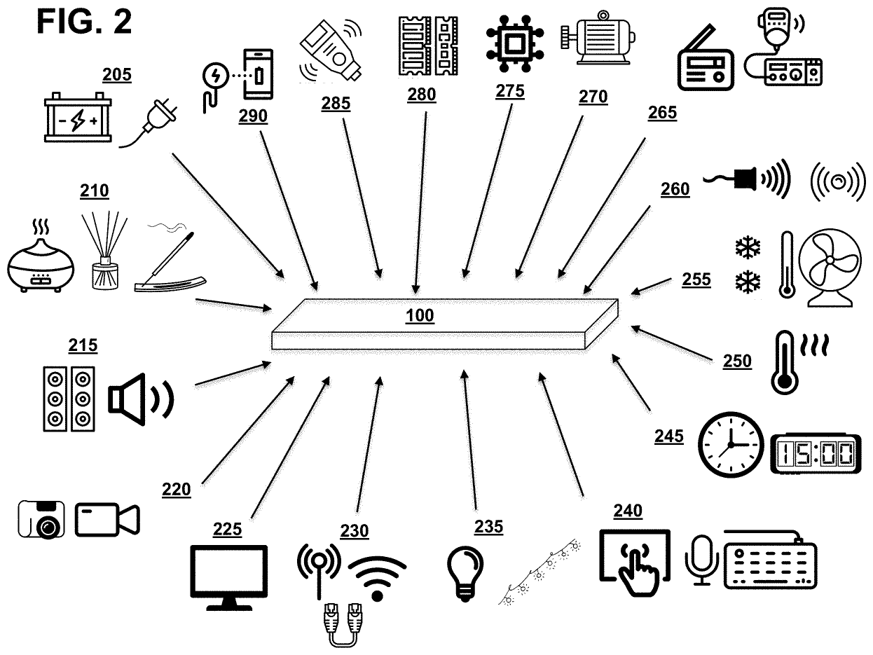

FIG. 2 discloses exemplary accessories 150 that may be incorporated into an exercise mat 100. Exemplary accessories 150 that may be integrated into an exercise mat can include, inter alia, one or more power sources 205, one or more aroma dispensers 210, one or more audio devices 215, one or more camera devices 220, one or more display devices 225, one or more communication interfaces or devices 230 (e.g., wired and/or wireless communication interfaces or devices), one or more lighting components 235, one or more input devices 240, one or more timepieces 245, one or more heating components 250, one or more cooling components 255, one or more sensors 260, one or more radio devices 265, one or more motors 270, one or more processors 275, one or more electronic storage devices 280, one or more massage components 285, and/or one or more wireless charging devices 290. Other types of accessories may also be integrated or incorporated into the exercise mats 100.

The aforementioned accessories, as well as any other accessories 150 mentioned in this disclosure, can be integrated directly or indirectly into the body portion 110 of the exercise mats 100. Integrating an accessory 150 into an exercise mat 100 can include: incorporating all or a portion of the accessory 150 within the body portion 110 of the exercise mat 100; incorporating all or a portion of the accessory 150 between a top surface 120 and a bottom surface 130 of the exercise mat; connecting or attaching the accessory to the body portion 110 of the exercise mat 100 (e.g., such that all or a portion of the accessory 150 extends into or through the body portion of the exercise mat 100); attaching or connecting the accessory 150 to a surface (e.g., a top surface 120, bottom surface 130, and/or side surface 140) of the exercise mat 100; incorporating a compartment, pocket, and/or sleeve that houses or receives the accessory 150 in the body portion 110 of the exercise mat 100; incorporating one or more connectors into the body portion 110 of the exercise mat 100 which can be used to connect or attach the accessory 150; connecting the accessory to one or more wired connection interfaces and/or one or more wireless communication interfaces included in the exercise mat 100; connecting an accessory dashboard comprising one or more accessories 150 to a body portion 110 of the exercise mat 100; and/or incorporating an accessory dashboard into a body portion of the exercise mat 100 (e.g., such that all or a portion of the accessory dashboard is located between a top surface and a bottom surface of the accessory mat). Accessories 150 may be integrated into the exercise mats 110 in other ways as well.

In certain embodiments, one or more power sources 205 may be directly or indirectly integrated or incorporated into the exercise mats 100 and/or connected to the exercise mats 100. Each power source 205 included in the exercise mat can include any type of alternating current (AC) power source and/or direct current (DC) power source. In certain embodiments, the one or more power sources 205 can include one or more batteries (e.g., rechargeable and/or non-rechargeable batteries). The one or more power sources 205 can additionally, or alternatively, include wires and/or plugs that can be connected to outlets. The one or more power sources 205 can be utilized to supply power to any other accessories 150 (e.g., electronic aroma dispensers, audio devices, display components, wireless chargers, etc.) incorporated into the exercise mats 100. For example, the one or more power sources 205 may be electrically connected to some or all of the accessories 150 incorporated or integrated into the exercise mat and can provide power to the accessories 150. The one or more power sources 205 may be electrically connected to the accessories 150 via one or more wires, cables, and/or circuits (which, in some cases, may be incorporated within the body portion 110 of the mat itself and/or a dashboard integrated with the mat). The one or more power sources 205 also can be utilized to charge electronic devices (e.g., such as mobile phones, digital media devices, MP3 players, and/or any other electronic devices) of individuals who are utilizing the exercise mat 100 (e.g., by connecting the electronic devices to a port or interface on the mat 100).

In certain embodiments, the exercise mat 100 and/or power sources 205 may include one or more power indicators that display information indicating the amount of power remaining in a battery (or batteries) incorporated into the mat 100. For example, a display may visually depict a percentage (e.g., 0%, 55%, 100%) indicating the remaining power. Additionally, or alternatively, the power indicators may include lights (e.g., LEDs) that can be used to indicate the amount of power remaining in the battery.

In certain embodiments, one or more aroma dispensers 210 may be directly or indirectly integrated or incorporated into the exercise mats 100 and/or connected to the exercise mats 100. Exemplary aroma dispensers 210 may include one or more oil diffusers, such as one or more ultrasonic diffusers, one or more humidifying diffusers, one or more nebulizing diffusers, one or more atomizing diffusers, one or more evaporative diffusers, one or more heat diffusers, one or more electronic diffusers, one or more electric diffusers, and/or other types of diffusers. These diffusers may be configured to diffuse or emit essential oils and/or other types of oils in ambient air or atmosphere in the vicinity of the exercise mats 100. In some cases, water may be added into the diffusers, and the diffusers can diffuse or emit a mist of combined water vapor and oil vapor. Other types of exemplary aroma dispensers 210 may include incense burners, reed diffusers, candles (included in candle holders integrated into the exercise mats), and/or aroma spraying devices.

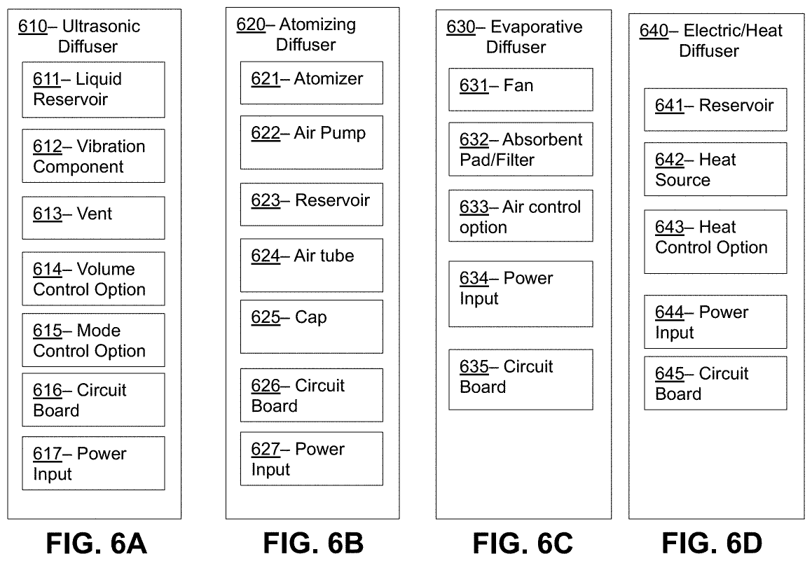

FIGS. 6A-6D disclose exemplary configurations of aroma dispensers 210, as well as exemplary components that may be included in the aroma dispensers 210.

As mentioned above, the exercise mats 100 may be equipped with, or connected to, one or more ultrasonic and/or humidifying oil diffusers (collectively referred to herein as ultrasonic oil diffusers 610). The configurations of the ultrasonic oil diffusers 610 may vary. In certain embodiments, each ultrasonic oil diffuser 610 creates a mist using an adiabatic process that does not require heat. For example, as shown in FIG. 6A, an ultrasonic diffuser 610 may represent an electronic device that includes a liquid reservoir 611 for storing or holding a mixture of water and oils (e.g., essential oils), a vibration component 612 (e.g., a metal diaphragm or disc which vibrates at an ultrasonic frequency) that breaks down the oils and/or water into micro-particles to create a mist (e.g., a very fine mist comprising oil and water particles), and a vent 613 that allows the mist to be dispersed into the air. The ultrasonic oil diffuser 610 may further include a volume control option 614 (e.g., a dial or control that permits a user to adjust the volume of mist that is dispensed), a circuit board 616, and a power input 617 (e.g., which may be connected to one or more power sources 205 of the exercise mat 100). The ultrasonic oil diffuser 610 also may include a mode control option 615 that enables the diffuser to operate in different output modes (e.g., a continuous output mode during which mist is continuously output or an intermittent mode in which mist is periodically output at different intervals). All or a portion of the diffuser components (e.g., the reservoir 611, the vibrating component 612, the vent 613, the volume control option614, mode control option 615, circuit board 616, power input 626, etc.) may be integrated or incorporated into the exercise mat 100. In some cases, some or all of the diffuser components can be situated adjacent to one another in a horizontal plane to minimize the sizes of the ultrasonic oil diffuser 610 and/or the exercise mat 100 integrated with the ultrasonic oil diffuser 610.

The exercise mats 100 can additionally, or alternatively, be equipped with, or connected to, one or more nebulizer diffusers and/or atomizing diffusers (collectively referred to herein as atomizing diffusers 620). The configurations of the atomizing diffusers 620 may vary. For example, as shown in FIG. 6B, an atomizing diffuser 620 may include an atomizer 621 that is configured to create a mist (e.g., without using water), which is then diffused into the ambient air in the vicinity of the device. In certain embodiments, the atomizing diffuser 620 may generate a stream of pressurized air to atomize oils into micro-particles and to create a mist.

For example, in certain embodiments, the atomizing diffuser 620 may include, inter alia, an air pump 622, a reservoir (e.g., a glass reservoir or air reservoir), an air tube 624 connected to the air pump 622 and extending to the reservoir 623, and a cap 625 (e.g., a glass cap, end cap, or nozzle). The air pump 622 can generate an air current that is directed through the air tube 624 and provided to the reservoir 623. When the air current is forced through the air tube 624, the velocity of the air current may increase while the pressure at the exit point is reduced. The difference in pressure can create a suction that causes oils (e.g., which may be included within a separate tube) to rise upwards. A stream of pressurized air may hit the oil and causes it to atomize into micro-particles that fill the reservoir and thereafter emitted out of the cap into the air. The nebulizer diffuser and/or atomizing diffuser may further include a volume control option, circuit board 626, and power input 627 (e.g., which may be connected to one or more power sources of the exercise mat). All or a portion of the diffuser components (e.g., the atomizer 621, air pump 622, reservoir 623, air tube 624, glass cap 625, circuit board 626, power input 627, volume control option, etc.) may be integrated or incorporated into the exercise mat. In some cases, some or all of the diffuser components can be situated adjacent to one another in a horizontal plane to minimize the sizes of the atomizing diffuser 620 and/or the exercise mat 100 integrated with the atomizing diffuser 620.

The exercise mats 100 additionally, or alternatively, can be equipped with, or connected to, one or more evaporative diffusers 630. The configurations of the evaporative diffusers 630 may vary. For example, as shown in FIG. 6C, an evaporative diffuser 630 may include one or more fans 631 that are used to covert oils to gas (e.g., by causing the oils to evaporate into the ambient air). In certain embodiments, the evaporative diffusers 630 also may include one or more absorbent pads or filters 632 that can absorb oil, and the one or more fans 631 can blow an air current onto the absorbent pads to convert the oil from liquid to gas. The evaporative diffusers 630 also may include an air control option 633 (e.g., which controls the power level of the one or more fans 631), a power input 634 (e.g., which may be connected to one or more power sources 205 of the exercise mat 100) for powering the fan and/or other components of the evaporative diffusers, and/or a circuit board. All or a portion of the diffuser components (e.g., the fan 631, absorbent pads 632, air control option 633, power input 634, circuit board 635, etc.) may be integrated or incorporated into the exercise mat 100. In some cases, some or all of the diffuser components can be situated adjacent to one another in a horizontal plane to minimize the sizes of the evaporative diffuser 630 and/or exercise mat 100 integrated with the evaporative diffuser 630.

The exercise mats 100 can additionally, or alternatively, be equipped with, or connected to, one or more heat diffusers and/or electric diffusers 640. The configurations of the heat and/or electric diffusers 640 may vary. For example, as shown in FIG. 6D, a heat and/or electric diffuser 640 can be configured to apply heat to oil included in a reservoir 641 (e.g., a tray) to convert the oil from liquid to gas and/or to evaporate the oil. In some cases, electricity and/or electrically powered heating sources 642 can be applied to heat the oil. A candle and/or other heat source 642 also can also be utilized to heat the oil. The heat diffusers and/or electric diffusers 640 may further include a heat control option 643 (e.g., which controls the intensity of the heat), a power input 644 (e.g., which may be connected to one or more power sources of the exercise mat) for powering the heat source and/or other components, and/or a circuit board 645. All or a portion of the diffuser components (e.g., the reservoir 641, heat source 642, heat control option 643, power input 644, circuit board 635, etc.) may be integrated or incorporated into the exercise mat 100. In some cases, some or all of the diffuser components can be situated adjacent to one another in a horizontal plane to minimize the sizes of the heat and/or electric diffuser 640 and/or exercise mat 100 integrated with the heat and/or electric diffuser 640.

Any of the aroma dispensers 210 that include a reservoir (e.g., a water and/or air reservoir) can allow the reservoir to be detached and/ore removed, thus allowing the reservoir to be cleaned and/or refilled. In certain embodiments, the exercise mat 100 may include an opening (e.g., a pocket, a compartment, and/or partition that can be sealed and unsealed) that enables the reservoir to be removed and re-inserted.

Moreover, any of the aroma dispensers 210 or diffusers can include adjustable nozzles that can be moved, rotated, and/or manipulated to control the direction of mist, evaporated oils, and/or air current originating from the diffusers. The configuration of the nozzles can vary. In certain embodiments, the adjustable nozzles can include ball-and-socket connectors that enable the nozzles to be manipulated in various directions. The nozzles may alternatively, or additionally, include other types of connectors, such as swivel connectors, rotational connectors, etc.

Any of the aroma dispensers 210 can include a volume control option that enables a user to change or adjust the volume of mist, vapor, and/or air current that is output by the diffuser. Any of the aroma dispensers 210 also can include a mode option that enables a user to change or adjust the mode of the diffuser (e.g., including a continuous mode, intermittent mode, etc.). Any of the aroma dispensers 210 that are electronic can include an automatic shut off feature that stops the aroma dispenser from functioning (e.g., from dispersing oil, vapor, mist, and/or air current). For example, an automatic shut off feature may be engaged in response to the aroma dispensers 210 detecting any or all of the following: 1) that there is no water in a water reservoir of the aroma dispenser; 2) that the water in a water reservoir of the aroma dispenser is below a certain threshold; and/or 3) overheating of the diffuser and/or component of the diffuser. The nozzles, volume control options, automatic shut off features, and other aroma dispenser features may be integrated or incorporated into the exercise mats 100.

In certain embodiments, at least one aroma dispenser 210 is integrated into an exercise mat 100, and the aroma dispenser 210 is configured to be detachable from the exercise mats 100. For example, the components of the aroma dispenser 210 can be included in a housing, and the aroma dispenser may include a connector (e.g., a female USB or mini-USB connector) that permits the aroma dispenser to be attached to a corresponding connector (e.g., a male USB connector) included on the exercise mats 100. In addition to physically securing the aroma dispenser 210 to the mat, the connector can provide power to the aroma dispenser (e.g., power from one or more power sources 205). The aroma dispenser 210 can be attached to exercise mat 100 when aromatherapy is desired, and detached to permit cleaning and/or refilling of the reservoir.

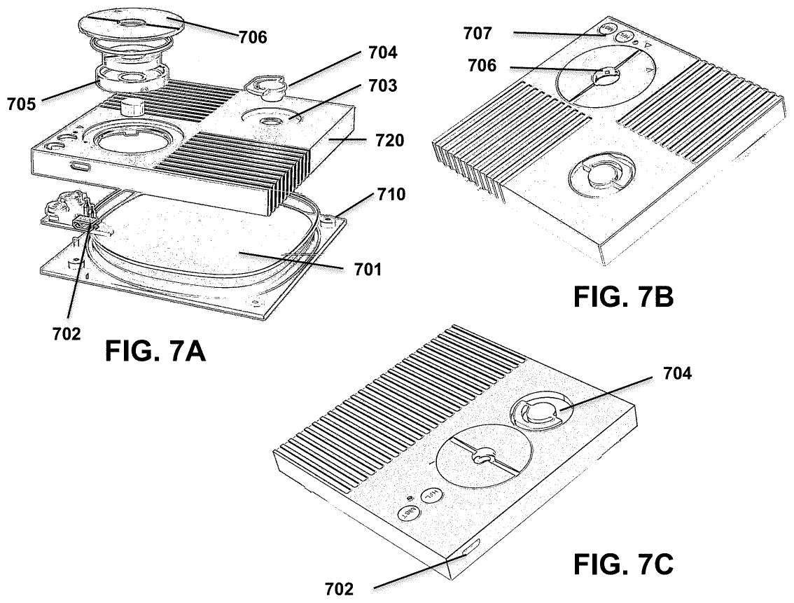

FIGS. 7A-7B disclose an exemplary embodiment of a compact ultrasonic diffuser 610. FIG. 7C is another exemplary ultrasonic diffuser 610 that has the same or similar internal configuration, but modified housing. These unique ultrasonic diffusers 610 are designed to be miniature and compact so that it does not protrude from the surface of exercise mats 100, and use micro-jet ultrasonic diffusion technology. Moreover, it is designed to be detached and reattached from the exercise mats 100.

The ultrasonic diffuser 610 includes a housing base portion 710 that comprises a water reservoir 701 and a connector 702 (e.g., mini USB connector for connecting to exercise mats 100 and power the device). It also a top housing porting 720 that includes an opening 703 (e.g., for inserting water and oils into the water reservoir), a removable plug 704 (e.g., for sealing and unsealing the opening 703), a vibration component 705 (e.g., converting the water and/or oils into vapor), an output 706 (e.g., for outputting the vapor), and one or more control options 707 (e.g., for activating/deactivating the device, adjusting the volume of vapor output, and changing operational modes). The vibration component 705 is located between an upper and lower cover as shown in FIG. 7A. In some cases, it also may include one or more circuit boards (e.g., for controlling the functions associated with the vibration component 705 and control options 707).

These components are arranged in a unique fashion to minimize the depth of the device, which in some cases may between 6 mm to 13 mm in depth (e.g., 8 or 9 mm). The majority of the components (including the opening 703, control options 707, and output 706) are located on a flat upper surface of the device to maximize the size of the water reservoir 701, and minimize the depth of the device. The length and width of the device can be approximately 80 mm to 90 mm (e.g., 85 mm) in some cases.

Returning to FIG. 2, in certain embodiments, one or more audio devices 215 may be directly or indirectly integrated or incorporated into the exercise mats 100 and/or connected to the exercise mats 100. The one or more audio devices 215 can include any type of speaker devices for outputting sound. The audio devices may additionally, or alternatively, include one or more microphones, one or more speakers, and/or one or more audio cards. The one or more audio devices 215 may be wirelessly coupled (e.g., via a Bluetooth and/or Wi-Fi connection) to an electronic device (e.g., mobile device, smart phone, desktop computer, etc.) of an individual utilizing the exercise mat 100, and may receive audio data from the electronic device. The one or more audio devices 215 can enable users to listen to audio (e.g., music and/or exercise routine instructions) while using the exercise mats 100. In certain cases, one or more audio devices 215 may be integrated into display devices 225 associated with the exercise mats 100. Any audio devices 215 included on an exercise mat 100 can be powered by one or more power sources 205 included on the exercise mat 100.

In certain embodiments, one or more camera devices 220 may be directly or indirectly integrated or incorporated into the exercise mats 100 and/or connected to the exercise mats 100. Exemplary camera devices 220 may include any type of analog or digital still image cameras and/or any type of analog or digital video cameras. Each camera device 220 may include one or more optical sensors for capturing image and/or video content and/or one or more audio sensors (e.g., microphones) for capturing audio content. Any camera devices 220 included on the exercise mat 100 can be powered by one or more power sources 205 included on the exercise mat 100. The one or more camera devices 220 may be wirelessly coupled (e.g., via a Bluetooth and/or Wi-Fi connection) to an electronic device (e.g., mobile device, smart phone, desktop computer, etc.) of an individual utilizing the exercise mat 100, and to permit the individual to control the camera devices 220 (e.g., viewing angle, filter options, position, etc.).

In certain embodiments, one or more camera devices 220 included on the exercise mats 100 may enable users to participate in live exercise sessions (e.g., yoga sessions, Pilates sessions, mediation sessions, workout sessions, etc.) by accessing an online platform. For example, in certain embodiments, users may access an online platform that provides live exercise sessions, and the users may utilize camera devices 220 included on the exercise mats 100 (and/or camera devices 220 included on mobile devices and/or electronic devices operated by the users) to participate in a one-way and/or bi-directional video conference sessions during the exercise sessions or while using the exercise mats 100 for other purposes.

In certain embodiments, any video and/or image content captured by the one or more camera devices may be stored on a storage device (e.g., such as a storage device 280 included on the exercise mat and/or a storage device associated with an online platform) for later retrieval and viewing. In some scenarios, users may utilize the one or more camera devices 220 to record exercise sessions and to view the recorded sessions at a subsequent times. This may allow the users to analyze their exercise routine and/or to improve exercise techniques. In certain embodiments, the image and/or video content may be analyzed with analysis software that is configured to automatically to provide feedback on exercise techniques (e.g., yoga poses).

In certain embodiments, one or more display devices 225 may be directly or indirectly integrated or incorporated into the exercise mats 100 and/or connected to the exercise mats 100. The one or more display devices 225 may be configured to receive audio/video (AV) data and output the AV data. The AV data output by the display devices 225 may include data that was captured by the camera devices 220, data that was received from an online platform (e.g., exercise sessions), and/or data that was received over a network (e.g., the Internet, a television network, etc.). The one or more display devices 225 may include video display devices, image display devices, television devices, monitors, screens (e.g., liquid crystal display (LCD) screens, plasma screens, etc.), cathode ray tube devices, and/or other similar types of devices. Any television device included on the exercise mat 100 may be connected to, and allow for bi-directional communication with, a television network. Any display device 225 included on the exercise mat 100 can be powered by one or more power sources 205 included on the exercise mat 100.

In certain embodiments, the one or more display devices 225 may be configured to display content captured by the one or more camera devices 220. For example, the one or more display devices 225 may permit users to view themselves in real-time as they perform exercise routines and/or they perform exercise routines. In certain embodiments, the one or more display devices 225 may be configured to display pre-recorded exercise routines (e.g., yoga routines, mediation routines, Pilates routines, weight training routines, etc.), which may or may not be accessed from an online platform. In certain embodiments, the one or more display devices 225 may be configured to display a live instructor (e.g. a live yoga or Pilates instructor) and/or other content associated with participating in a live exercise session.

In certain embodiments, one or more communication devices and/or interfaces 230 may be directly or indirectly integrated or incorporated into the exercise mats 100 and/or connected to the exercise mats 100. The one or more communication devices and/or interfaces 230 may include wired and wireless communication devices and/or interfaces that enable the exercise mats 100 to communicate using wired and/or wireless communication techniques. The exercise mats 100 may include any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can comprise Bluetooth, Zigbee, Wireless Universal Serial Bus (USB), Z-Wave, etc. Exemplary LAN and/or WAN protocol(s) can comprise Institute of Electrical and Electronic Engineers (IEEE) 802.3 (also known as Ethernet), IEEE 802.11 (also known as WiFi), etc. Exemplary wireless cellular network protocol(s) can comprise Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware included in the exercise mats 100 can depend on the network topologies and/or protocols implemented. In certain embodiments, exemplary communication hardware can comprise wired communication hardware including, but not limited to, one or more data buses, one or more universal serial buses (USBs), one or more networking cables (e.g., one or more coaxial cables, optical fiber cables, twisted pair cables, and/or other cables). Further exemplary communication hardware can comprise wireless communication hardware including, for example, one or more radio transceivers, one or more infrared transceivers, etc. Additional exemplary communication hardware can comprise one or more networking components (e.g., modulator-demodulator components, gateway components, etc.).

In certain embodiments, the one or more communication devices and/or interfaces 230 can include one or more transceiver devices, each of which includes a transmitter and a receiver for communicating wirelessly. The one or more communication devices and/or interfaces 230 also can include one or more wired ports (e.g., Ethernet ports, USB ports, auxiliary ports, etc.) and related cables and wires (e.g., Ethernet cables, USB cables, auxiliary wires, etc.).

In certain embodiments, the one or more communication devices and/or interfaces 230 incorporated into the exercise mats 100 also can include one or more modem devices, router devices, access points, and/or mobile hot spots. For example, modem devices may enable the exercise mats 100 (and/or accessories 150 included on the exercise mats 100) to be connected to the Internet and/or other network. The modem devices can permit bi-directional communication between the exercise mats 100 and the Internet (and/or other network). In certain embodiments, one or more router devices and/or access points may enable accessories 150 (and/or electronic devices operated by individuals) to be connected to a LAN and/or other more other networks. In certain embodiments, one or more mobile hot spots included in the exercise mats 100 may be configured to establish a LAN (e.g., a Wi-Fi network) that is linked to another network (e.g., a cellular network). The mobile hot spot may enable accessories 150 included on the exercise mat 100 (and/or electronic devices operated by individuals) to access the Internet and/or other networks. Any communication devices and/or interfaces 230 included on the exercise mat 100 can be powered by one or more power sources 205 included on the exercise mat 100.

In certain embodiments, the communication devices and/or interfaces 230 can be permit an exercise mat 100 to be coupled to a device (e.g., a smart phone, computer, tablet, etc.), and the device may enable a user to control any of the accessories 150 integrated with the exercise mat 100. For example, the device may present one or more GUIs that enable the users to control the audio components 215 (e.g., to stream audio to the audio components 215, adjust volume settings, etc.), the camera devices 220 (e.g., to adjust viewing angle, filters, recording options, etc.), and/or any other accessories 150 described herein.

In certain embodiments, one or more one or more lighting components 235 may be directly or indirectly integrated or incorporated into the exercise mats 100 and/or connected to the exercise mats 100. Exemplary lighting components 235 may include light-emitting diodes (LEDs), light bulbs, incandescent lighting components, fluorescent lighting components, tungsten-halogen lighting components, high-intensity discharge lighting components, and/or other types of light sources. In certain embodiments, the exercise mats and/or lighting components 235 may include control options that are able to switch the lighting components on/off and to select various lighting effects (e.g., strobe effects, glowing effects, dimming effects, fluctuating effects, etc.). In certain embodiments, the lighting components 235 can be used to indicate the remaining battery life of a power source 205 and/or to indicate if one or more accessories 150 are activated or turned on. Any lighting component 235 included on the exercise mat 100 can be powered by one or more power sources 205 included on the exercise mat 100.

In certain embodiments, one or more input devices 240 may be directly or indirectly integrated or incorporated into the exercise mats 100 and/or connected to the exercise mats 100. The input devices 240 can include one or more of the following: touchpads, touchscreens, keyboards, microphones, digital cameras, digital video cameras, camcorders, AN recorders, video cards, joysticks, mouse devices, buttons, switches, dials, and/or other devices. The input devices 240 can allow users to control the functionality and settings of any accessory 150 described in this disclosure, including any accessory 150 incorporated into the exercise mats 100 and/or in communication with the exercise mats 100. For example, the input devices may enable users to control aroma diffusers 210 (e.g., to activate/deactivate the devices, control output volume of mist or air current, select operational modes such as continuous or intermittent diffusion settings, etc.), audio devices 215 (e.g., to activate/deactivate the devices, select music or audio to be output, control volume settings, etc.), and other accessories 150 associated with the exercise mats 100.

The input devices 240 also can enable users to communicate with online platforms over a network. For example, the input devices 240 can enable users to select, download and/or view content (e.g., pre-recorded instructional exercise routines) from the online platforms, select live exercise sessions to participate in, and access various information stored on the online platform (e.g., recordings of the user performing exercises, health information tracked by the electronic mats, analytics generated by the online platform, etc.). Any input devices 240 included on the exercise mat 100 can be powered by one or more power sources 205 included on the exercise mat 100.

In certain embodiments, one or more timepieces 245 may be directly or indirectly integrated or incorporated into the exercise mats 100 and/or connected to the exercise mats 100. The timepieces 245 can include any analog and/or digital clock, watch, or device that indicates that the current time and/or date. In some cases, the timepieces 245 may be integrated into display devices 225 included on the exercise mats 100. Control options can be included on timepieces 245 and/or exercise mats 100 that enable users to adjust the current time and/or date that is displayed, set timers or alarms, and/or perform other related functions. Any timepiece 245 included on the exercise mat 100 can be powered by one or more power sources 205 included on the exercise mat 100.

In certain embodiments, one or more one or more heating components 250 may be directly or indirectly integrated or incorporated into the exercise mats 100 and/or connected to the exercise mats 100. The heating components 250 may be configured to generate heat and/or thermal radiation and apply the heat to users of the exercise mats 100. Exemplary heating components 250 may include infrared (IR) heating components, electric heating components, convention heating components, and/or other heating components. In certain embodiments, the exercise mats 100 and/or heating components 250 may include control options that enable users to control or adjust the amount or amount of heat that is output. Any of the heating components 250 included on the exercise mat 100 can be powered by one or more power sources 205 included on the exercise mat 100.

In certain embodiments, one or more one or more cooling components 255 may be directly or indirectly integrated or incorporated into the exercise mats 100 and/or connected to the exercise mats 100. The cooling components 255 can include any device or apparatus that can assist with cooling users of the exercise mats 100, generating an air current, and/or lowering ambient temperatures in vicinities of the exercise mats. Exemplary cooling devices can include fans and/or air conditioning devices. Other types of cooling devices can include cooling pads, ice packs and/or cooling packs. Any of the cooling components 255 incorporated into an exercise mat 100 can be powered by one or more power sources 205 included on the exercise mat 100.

In certain embodiments, one or more sensors 260 may be directly or indirectly integrated or incorporated into the exercise mats 100 and/or connected to the exercise mats 100. Exemplary sensors 260 included in the exercise mats 100 can include one or more of the following: heartbeat sensors, touch sensors, magnetic contact sensors, heat sensors, gas sensors, smoke sensors, pressure sensors, contact sensors, infrared (IR) sensors, proximity sensors, light sensors, temperature sensors, acoustic sensors, sound sensors, video sensors, imaging sensors, seismic sensors, and/or other types of sensors. The sensors 260 may be incorporated into various accessories 150 (e.g., a video sensor may included a camera device 220, an audio sensor may be included in an audio device 215, a touch sensor may be included in an input device 240, etc.) and/or may be separately or independently incorporated into the exercise mats 100. The sensors included in the exercise mats 100 can be used to perform various functions (e.g., monitoring health and vital information of users, detecting when diffusers should be turned off, capturing audio and/or video content, measuring body temperatures of users, monitoring heartbeats of users, detecting overheating of lighting components 235 and/or heating components 250, etc.). In certain embodiments, one or more of the aforementioned sensors (e.g., pressure, video, and/or image sensors) can be configured to monitor and track users' movements and/or stances (e.g., yoga poses) on the exercise mats, and/or monitor and track the movements and/or stances of the users themselves. In certain embodiments, the sensors may be arranged in matrices and embedded between top and bottom surfaces of the exercise mats to track user movements. Any of the sensors 260 incorporated into the exercise mat 100 can be powered by one or more power sources 205 included on the exercise mat 100.

In certain embodiments, one or more radio devices 265 may be directly or indirectly integrated or incorporated into the exercise mats 100 and/or connected to the exercise mats 100. The radio devices 265 may include one or more amplitude modulation (AM) radio devices, frequency modulation (FM) radio devices, satellite radio devices, high-definition (HD) radio devices, and/or other types of radio devices. Any satellite radio devices and/or HD radio devices may be connected to, and allow for bi-directional communication with, a radio network. The radio devices 265 may further include radio receivers and transmitters associated AM radio devices, FM radio devices, satellite radio devices, HD radio devices, and/or other radio devices. The radio devices 265 can enable users to listen to radio programs and/or other audio while using the exercise mats 100. Any of the radio devices 265 incorporated into the exercise mat 100 can be powered by one or more power sources 205 included on the exercise mat 100.

In certain embodiments, one or more motors 270 may be directly or indirectly integrated or incorporated into the exercise mats 100 and/or connected to the exercise mats 100. The one or more motors 270 can include any type of motor including, but not limited to, electric motors, servomotors, and/or combustion motors. The one or more motors 270 can be used to power one or more accessories 150 included on the exercise mats. For example, one or more motors 270 may be used to power aroma dispensers 210 (e.g., oil diffusers), heating components 250, cooling components 255, massage components 285, and/or other accessories 150. The motors 270 may be incorporated into the accessories 150 themselves and/or may be separately or independently incorporated into the exercise mats 100. Some or all of the motors 270 incorporated into the exercise mat 100 can be powered by one or more power sources 205 included on the exercise mat 100.

In certain embodiments, one or more processors 275 may be directly or indirectly integrated or incorporated into the exercise mats 100 and/or connected to the exercise mats 100. The one or more processors 275 can include any or all of the following: central processing units (CPUs), graphics processor units (GPUs), controllers, microprocessors, digital signal processors, computational circuits, microcontrollers, controllers, complex instruction set computing (CISC) microprocessors, reduced instruction set computing (RISC) microprocessors, very long instruction word (VLIW) microprocessors, digital signal processors, and/or any other type of processor or processing circuit capable of performing desired functions. The one or more processors 275 may also include application specific integrated circuits (ASICs) can be programmed to carry out any of the functions described herein. Any of the processors 275 incorporated into the exercise mat 100 can be powered by one or more power sources 205 included on the exercise mat 100.

In certain embodiments, one or more storage devices 280 may be directly or indirectly integrated or incorporated into the exercise mats 100 and/or connected to the exercise mats 100. The one or more storage devices 280 may communicate with the one or more processors 275, and the one or more processors 275 can execute any instructions stored on the one or more storage devices 280. The one or more storage devices 280 may include: i) non-volatile memory, such as, for example, read only memory (ROM) or programmable read only memory (PROM); and/or (ii) volatile memory, such as, for example, random access memory (RAM), dynamic RAM (DRAM), static RAM (SRAM), etc. In certain embodiments, the one or more storage devices 280 can comprise (i) non-transitory memory and/or (ii) transitory memory. Any of the storage devices 280 incorporated into the exercise mat 100 can be powered by one or more power sources 205 included on the exercise mat 100.

The one or more processors 275 can be configured to perform any of the exercise mat functionality described herein. For example, the one or more processors 275 may be configured to execute instructions for controlling any of the accessories 150 (and any of their functionalities) described herein and/or communicating with online platforms (including any interactions with online platforms described in this disclosure). Some or all of the functions executed by the one or more processors 275 can be based on instructions stored on the storage devices 280.

In certain embodiments, one or more massage components 285 may be directly or indirectly integrated or incorporated into the exercise mats 100 and/or connected to the exercise mats 100. The massage components 285 can include any device or component that is capable of massaging a portion of a user's body (e.g., back, arms, legs, head, etc.). In some embodiments, the massage components 285 can include one or more vibrating components that massage all or a portion of a user's body. The massage components 285 can additionally, or alternatively, include one or more mechanical massage components (e.g., which may rotate and/or apply pressure to provide a massaging experience), one or more massage rollers, and/or on or more motors 270 (e.g., which can vibrate to provide massaging effects). Any of the massage components 285 incorporated into the exercise mat 100 can be powered by one or more power sources 205 included on the exercise mat 100.

In certain embodiments, one or more wireless charging devices 290 may be directly or indirectly integrated or incorporated into the exercise mats 100 and/or connected to the exercise mats 100. The one or more wireless charging devices 290 may be configured to wirelessly charge (without a cable or wire connection) various electronic devices (e.g., mobile phones, tablets, digital media players, and/or other electronic devices) and/or one or more accessories 150 included on the exercise mat 100. In certain embodiments, the wireless charging devices 290 included in the exercise mat 100 can be configured to transfer energy to receivers included in the electronic devices and/or accessories 150 using electromagnetic induction. In some cases, the wireless charging devices 290 use one or more induction coils to create an alternating electromagnetic field, which interacts with receiver coils (included in the electronic devices and/or accessories 150) to generate electricity that can be transferred into batteries included in the electronic devices and/or accessories 150. Any of the wireless charging devices 290 incorporated into the exercise mat 100 can be powered by one or more power sources 205 included on the exercise mat 100.

In certain embodiments, the electronic devices (e.g., mobile phones, tablets, smart watches, and/or other electronic devices associated a user of the exercise mat) and/or one or more accessories 150 included on the exercise mat 100 can be charged using a hardwired connection. For example, the electronic devices and/or accessories 150 can be charged by connecting them to one or more USB ports and/or other ports (e.g., micro-USB ports, auxiliary ports, etc.) included on the exercise mat 100. The ports may be connected to one or more power sources 205 included on the exercise mat 100, which permit the electronic devices and/or accessories 150 to be charged.

In certain embodiments, the one or more controllers may be directly or indirectly integrated or incorporated into the exercise mats 100 and/or connected to the exercise mats 100. The one or more controllers may be implemented in hardware, software, and/or a combination of the two. In certain embodiments, the controller can be coupled to some or all of the accessories 150 included on the exercise mat 100, and can be configured to control any and all functionality associated with each of the accessories 150 (including, but not limited to, any of the functions described in this disclosure.

For example, in scenarios where one or more diffusers are incorporated into the exercise mats 100, the controller can be configured to activate/deactivate the one or more diffusers and adjust the output volume of the one or more diffusers. Similarly, in scenarios where one or more audio devices 215 are incorporated into the exercise mats 100, the controller can be configured to activate/deactivate the audio devices 215, select the audio (e.g., music) that is output, the volume of the audio, etc.

In certain embodiments, the controllers may be in communication with one or more input devices 240 that enable individuals using the exercise mats to control the functionalities of the accessories 150. For example, the one or more input devices 240 may include touch screen devices, knobs, dials, switches, and/or other similar devices. The input devices 240 may send signals to the controller identifying the inputs or options selected by a user, and the controller may transmit signals to the accessories 150 to control the functionalities of the accessories 150. In some embodiments, the input devices 240 and/or controller may enable the individuals to control the functionalities of the aroma dispensers 210, audio devices 215, camera devices 220, cooling components 255, heating components 250, etc. The input devices 240 and/or controller may also enable the users to select and control various functions associated with communicating with an online platform over a network (e.g., accessing pre-recorded sessions, participating in live sessions, transmitting tracking information to the online platform, etc.).

In certain embodiments, the exercise mats 100 may permit any accessory 150 integrated or incorporated into the exercise mats 100 to be detached and reattached as desired. This can be advantageous in scenarios where individuals wish to utilize the exercise mats 100 without the accessories 150. The connections between the accessories 150 and an exercise mat 100 can be releasable connections that enabled the accessories to be physically decoupled and/or electrically decoupled from the exercise mat 100 when desired.

In certain embodiments, the releasable connections on the exercise mats 100 may include connectors (e.g., USB ports, micro USB ports, auxiliary ports, etc.) that enable accessories 150 to be interchanged. For example, the accessories 150 may each include a common connector (e.g., the same type of electrical connector) that is able to be connected to a corresponding connector on the exercise mat 110, thus allowing the accessories to be swapped as desired by users. The types of connectors may vary. In some cases, the accessories 150 and exercise mats may include corresponding portions of a USB connection, micro-USB connection, auxiliary connection, and/or other types of connections. Regardless of the type of connectors that are incorporated, the connection between the accessories 150 and the exercise mat 150 may permit the accessories to be electrically powered and/or to communicate with other components included on the exercise mat 100 (e.g., such as power sources 205, processors 275, input devices 240, etc.).

In certain embodiments, the connectors (e.g., ports or electrical connections) may be included in recesses, openings, and/or holes located on, or incorporated into, a top surface of the exercise mat 100. When the accessories 150 are connected to the exercise mat 100, the accessories may be placed into the recesses. In some scenarios, when an accessory 150 is inserted into a recess included on the top surface of the exercise mat 100, the top most portion of the accessory does not extend above the top surface of the exercise mat 100 to avoid interference with usage of the exercise mat by users. In other scenarios, when an accessory 150 is inserted into a recess included on the top surface of the exercise mat 100, the top most portion of the accessory may extend above the top surface of the exercise mat 100, but at least a portion of the accessory 150 is hidden or included within the recess.

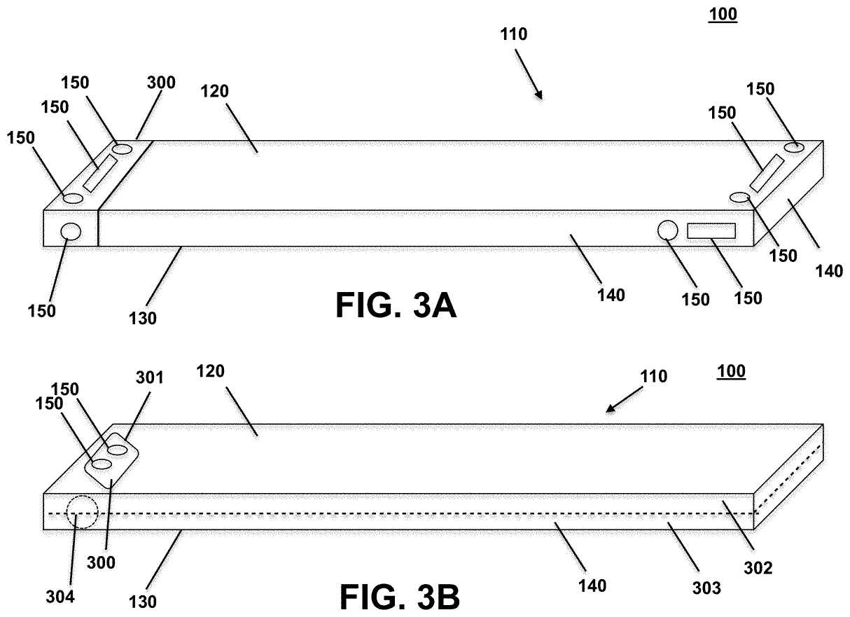

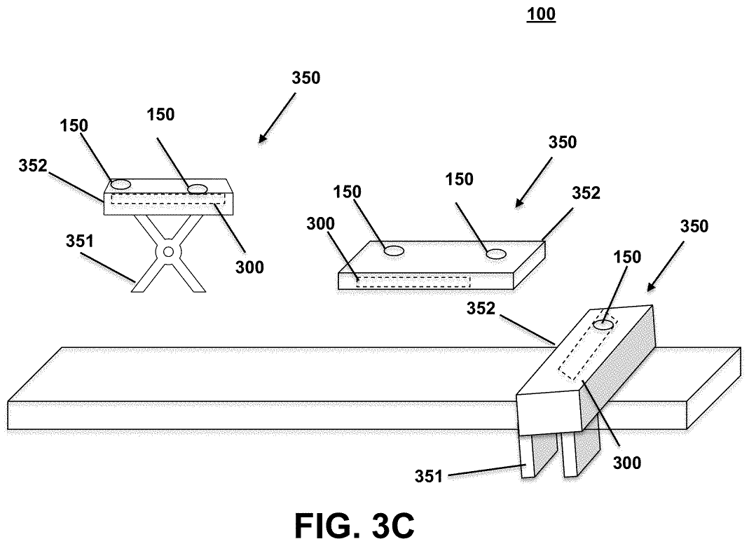

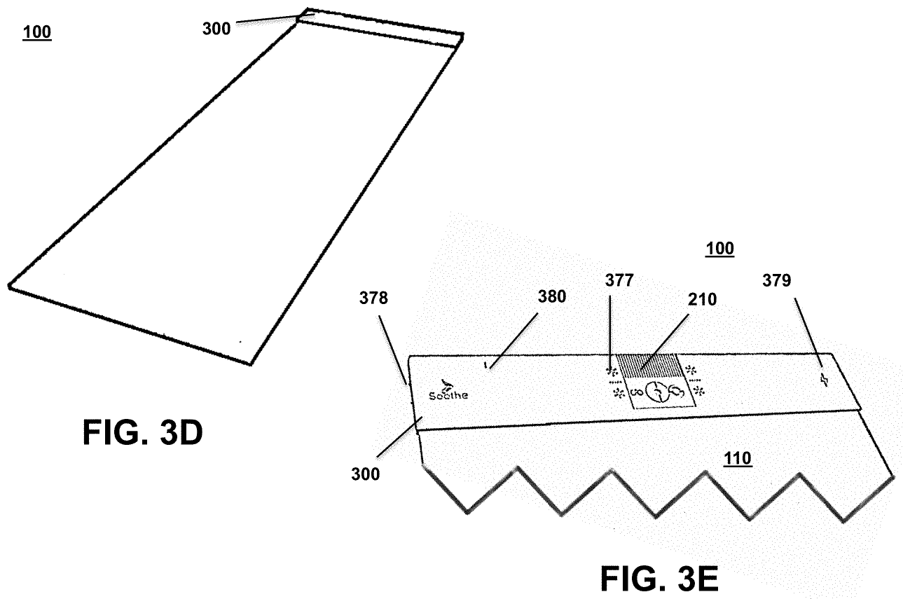

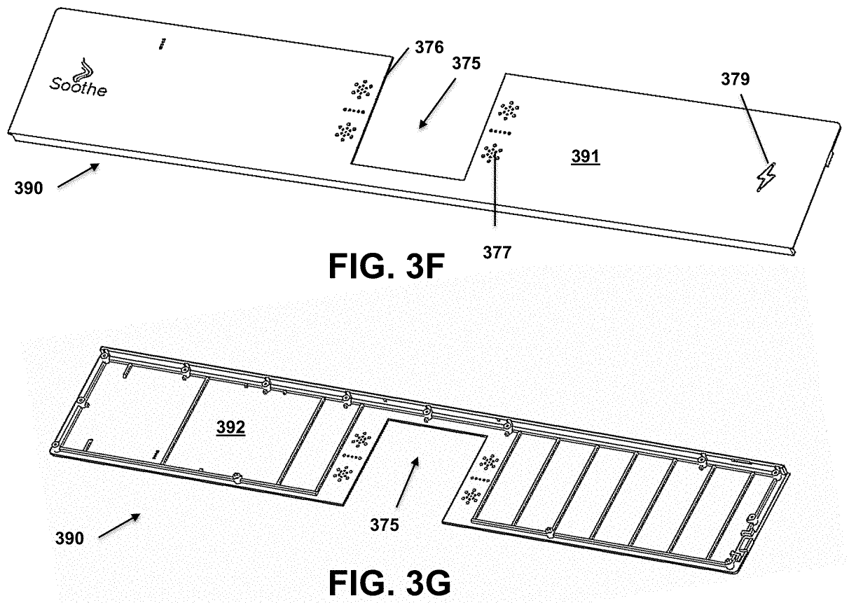

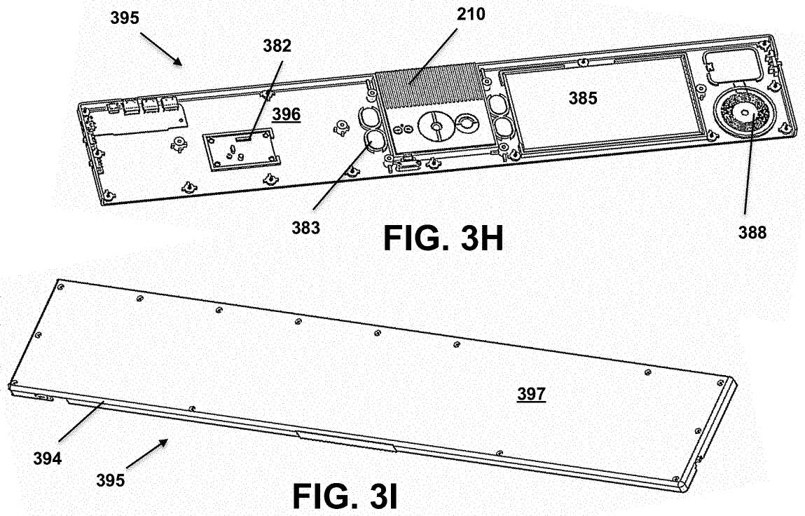

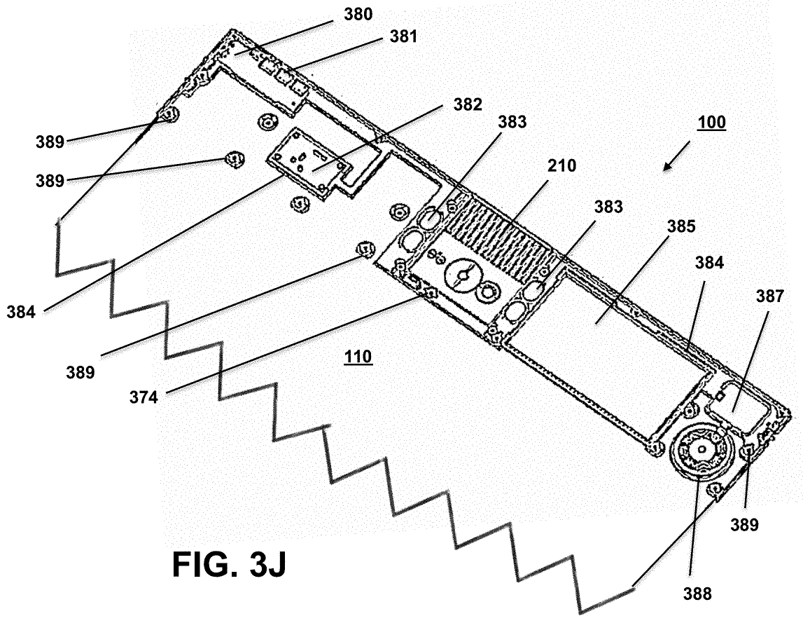

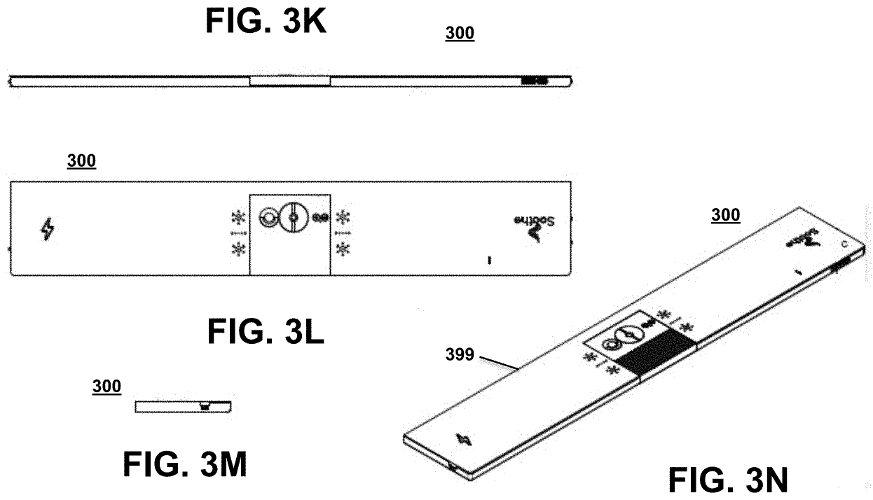

FIGS. 3A-3C disclose exemplary exercise mats 100 that include an accessory dashboard 300 comprising one or more accessories 150. The accessory dashboard 300 may represent an apparatus, housing, device, and/or component that houses or integrates one or more accessories 150 into a single structure, and/or which includes or is connected to a power source 205 capable of powering some or all of the accessories 150 included in the structure. The accessories 150 incorporated into the accessory dashboard 300 can include any combination of the accessories 150 described in this disclosure. The accessory dashboard 300 may include wires, circuitry, and/or other hardware for connecting each of the accessories 150 within the dashboard 300 to the power source 205 and/or to each other. The accessory dashboard 300 also may include wires, circuitry, and/or other hardware that permit exchange of data and/or information among the accessories 150. The accessory dashboard 300 also may include one or more input devices 240 that enable users to control the accessories 150 included in the accessory dashboard 150.

In one example, an accessory dashboard 300 may include a housing or structure that integrates various accessories 150 such as, for example, one or more aroma dispensers 210 (e.g., one or more oil diffusers), one or more audio devices 215 (e.g., one or more Bluetooth-enabled speaker), one or more input devices 240 (e.g., one or more touch screen devices, switches, dials, etc.), one or more wireless chargers 290 (e.g., one or more wireless charging coils), and one or more power sources 205 (e.g., one or more rechargeable batteries) that supply power to the accessories 150 integrated in the accessory dashboard 300 (e.g., the aroma dispensers 210, audio devices 215, wireless chargers 290, and input device 240). The accessory dashboard 300 may include wiring for electrically coupling the at least one aroma dispenser 210, audio device 215, wireless charger 290, and input device 240 to the power source 205. The accessory dashboard 300 may include appropriate circuitry and connections that enable the input device 240 to control the functionality of the at least one aroma dispenser 210, audio device 215, wireless charger 290, and power source 205.

In certain embodiments, it can be advantageous to incorporate one or more accessories 150 into the exercise mat 100 using an accessory dashboard 300 because the accessory dashboard 300 can include a rigid structure that protects the accessories 150 from damage. It also can be advantageous to incorporate one or more accessories 150 into the exercise mat 100 using an accessory dashboard 300 because doing so can enable the exercise mats 150 to be manufactured and/or fabricated more easily. For example, in some embodiments, the body portion 110 of the exercise mat 100 and the accessory dashboard 300 can be manufactured separately, and the accessory dashboard 300 can then be attached to the exercise mat 100 and/or integrated into the body portion of the exercise mat 100 to enhance the functionality of the exercise mat 100. In some cases, manufacturing the exercise mat 100 in this manner can be faster, more efficient, and less costly.

In FIG. 3A, the accessory dashboard 300 is attached to an end (e.g., a side surface 140) of the exercise mat 100. In certain embodiments, the accessory dashboard 300 maybe attached to the exercise mat 100 using adhesives (e.g., one or more glues), stitching, connectors (e.g., zippers, corresponding male/female connection pieces, screws, bolts, nuts, magnets, etc.), welding, soldering, and/or other attachment mechanisms. In certain embodiments, a portion of the accessory dashboard 300 located adjacent to the exercise mat 100 may include a recess or opening that is sized to receive an end of the body portion 110. The end of the body portion 110 may extend into the recess or opening, and the accessory dashboard 300 can be secured to the body portion 110 located inside of the recess or opening.

In certain embodiments, the accessory dashboard 300 can be attached to the exercise mat in a permanent or non-releasable fashion. In other embodiments, the accessory dashboard 300 can be a removable and/or de-attachable component that can easily be attached and detached form an exercise mat 100. In some cases, the latter can beneficial because an individual may desire the functionalities provided by the accessory dashboard 300 in some scenarios (e.g., when using the exercise mat at home or outside), but may not desire the functionalities of the accessory dashboard 300 in other scenarios (e.g., when attending an exercise class where in may not be appropriate to use aroma diffusers, play music, etc.). Thus, in certain embodiments, the user can attach the accessory dashboard 300 to the exercise mat 300 when desired, and can detach the accessory dashboard 300 when it is not desired. For embodiments in which the exercise mats 100 do not include accessory dashboards 300, each of the accessories 150 included on the mat can be configured to be removable or detachable for the same reasons.

For embodiments in which the accessory dashboard 300 is configured to be removable and/or detachable component, the accessory dashboard 300 and/or exercise mat 100 can include a releasable connection that permits attaching and detaching of the accessory dashboard 300 when desired. Exemplary releasable connections can include hook-and-loop fasteners (e.g., Velcro fasteners), releasable brackets, releasable snap connectors, releasable suction connectors, releasable zipper connectors, releasable button connectors, releasable male/female connectors, releasable hardware connectors (e.g., bolts, screws, and/or nuts), etc.

In certain embodiments, the accessory dashboard 300 can be designed such that it permits an existing or traditional exercise mat (e.g., without any of the accessories 150 described herein) to be retrofitted or upgraded with enhanced features, such as those performed by the accessories 150. In some cases, because traditional exercise mats can vary in size (e.g., can have varying widths, lengths, and heights/thicknesses), the structure of the accessory dashboard 300 can be configured such that it is able to expand and contract so that it can accommodate various sizes. For example, in certain embodiments, the accessory dashboard 300 may include an opening that receives an end portion of the exercise mat, and the structure of the accessory dashboard 300 permits the opening to expand and/or contract to accommodate varying widths and thicknesses of the exercise mats being received in the opening. The end portion of the exercise mat be inserted into the opening and secured to the accessory dashboard 150 in the opening.

In FIG. 3B, the accessory dashboard 300 is integrated into the body portion 110 of the exercise mat 100 and an opening 301 in the top surface 120 of the exercise mat 100 provides access to a portion of the accessory dashboard 300. One or more accessories 150 included on, or integrated into, the accessory dashboard 300 may be accessed via the opening 301. For example, in certain embodiments, the body portion 110 of the exercise mat 110 may include an upper portion 302 and a lower portion 303 that are connected, attached, and/or fused together during fabrication of the exercise mat 100. Prior to connecting the upper portion 302 and the lower portion 303, the accessory dashboard 300 may be inserted between the upper portion 302 and the lower portion 303. A pre-cut opening 301 in the top surface of the upper portion 302 can provide access to a portion of the accessory dashboard 300 and/or one or more accessories 150 included on the accessory dashboard 300.

In certain embodiments, the accessory dashboard 300 integrated into the body portion 110 of the exercise mat 100 can be permanently or non-releasably included within the body of the exercise mat 100. In other embodiments, the accessory dashboard 300 is removable. For example, the accessory dashboard 300 may be removed (and re-inserted when desired) via a second opening 304 included on the structure of the exercise mat 100. The opening 304 may be located on a top surface 120, bottom surface 130, and/or side surface 140 of the exercise mat 100.

Regardless of how the accessory dashboards 300 are integrated into the exercise mats 100, each accessory dashboard 300 can include any of the accessories 150 described in this disclosure including, but not limited to, one or more power sources 205, one or more aroma dispensers 210, one or more audio devices 215, one or more camera devices 220, one or more display devices 225, one or more communication interfaces 230, one or more lighting components 235, one or more input devices 240, one or more timepieces 245, one or more heating components 250, one or more cooling components 255, one or more sensors 260, one or more radio devices 265, one or more motors 270, one or more processors 275, one or more electronic storage devices 280, one or more massage components 285, one or more wireless charging devices 290, and/or one or more controllers.

In certain embodiments, the accessory dashboard 300 at least includes a power source 205 and the power source 205 may be used to power any of the accessories 150 integrated into accessory dashboard 300 (e.g., which, in some cases, may be a single accessory 150 and, in other cases, multiple accessories 150). The accessory dashboard 300 may include a housing that provides a structure for physically incorporating the accessories 150 and electrically connecting some or all of the accessories 150 to the power source 205. The housing may also include one or more input devices 240 that enable users to control the functions of the accessories 150 (including, but not limited to, any of the functions described in this disclosure).

It should be noted that any feature described herein as being incorporated into an exercise mat 100 can additionally, or alternatively, be incorporated into the accessory dashboards 300 described herein (including any accessories 150, accessory components, connections among accessories and/or their components, etc.).

In certain embodiments, accessory dashboards 300 can be incorporated into various structures that are physically separate from the exercise mats 100. For example, an accessory dashboard 300 may be integrated with a table, tray, stool, and/or other structure in some cases. These structures can be portable in nature and can be placed in the vicinity of exercise mats during use.

FIG. 3C discloses an exemplary embodiment of an accessory dashboard 300 that is incorporated into exemplary standalone structures 350, including an end table, a first tray (with no legs), and a second tray (with a pair of legs 351 situated on each side of an exercise mat 100). In these examples, the accessory dashboard 300 is incorporated into and/or included within a body portion 352 of the standalone structures 350, and accessories 150 included on the accessory dashboard 300 are accessible on a top surface of the body portion 352. In other embodiments, the accessories 150 may be directly integrated or incorporated into the body portions 352 of the standalone structures 350, and the accessories can be connected within the body portion 352 in the same manner as they can be in an accessory dashboard (e.g., with appropriate connections, circuitry, wiring, etc.). In the latter case, the body portion 352 of the standalone structures 350 may represent accessory dashboards 300 themselves.

The accessory dashboard 300 integrated into these standalone structures 350 can include any of the accessories 150 mentioned in this disclosure, and any of the accessory dashboard features mentioned in this disclosure can apply to the accessory dashboards 300 that are included in the standalone structures 350. In some cases, the standalone structures 350 may include one or more support structures 351 (e.g., legs), and the support structures may be collapsible. In certain embodiments, the support structures 351 can be collapsed or moved into a recess included on a bottom surface of the body portions 170 to provide a space-saving configuration. The accessory dashboard 300 and accessories 150 can be incorporated into other types of standalone structures 350 other then those depicted and described in this disclosure.

FIG. 3D discloses another exemplary configuration of an accessory dashboard located an end of an exercise mat 100 and extended the width of the exercise mat.

FIGS. 3E-3J disclose another exemplary configuration of an accessory dashboard 300 according to certain embodiments. In this embodiment, the accessory dashboard 300 includes a two-piece housing, including an upper housing portion 390 and a lower housing portion 395. The upper housing portion 390 includes a top surface 391 and a bottom surface 392, and the lower housing portion 395 a top surface 396 and a bottom surface 397. The upper housing portion 390 and lower housing portion can be connected to each other using one or more mechanical connectors (e.g., screws 389 or bolts).

An aroma dispenser 210 (e.g., an ultrasonic diffuser 610, atomizing diffuser 620, evaporative diffuser 630, and/or electric or heat diffuser 640) is included near the center of the accessory dashboard 300. In this example, the aroma dispenser 210 includes a housing that is separate from the housing of the accessory dashboard 300, and the housing of the aroma dispenser can include various subcomponents (e.g., such as a reservoir, vibration component, air pump, and/or heat source). The aroma dispenser 210 can be detached from, and reattached to, the accessory dashboard 300 (e.g., to permit refilling with oils and water, and to allow for cleaning). For example, a top surface of the accessory dashboard 300 can include a cutout 375 or opening for physically receiving and attaching the aroma dispenser 210 (e.g., the aroma dispensers illustrated in FIGS. 7A-7C). When inserted into the cutout 375 or opening, outer portions of the aroma dispenser 210 may be located beneath the edge 376 to secure the aroma dispenser 210 in place.

The accessory dashboard 300 also may include an electrical connector 374 (e.g., a mini USB female connector) that is configured to mate with, and electrically couple with, a corresponding electrical connector (e.g., a mini USB male connector) included on the aroma dispenser 210. This electrical connection enables the aroma dispenser 210 to be powered by one or more power sources 205 (e.g., one or more batteries 385) included within the accessory headboard 300. This electrical connection also enables the aroma dispenser 210 to be powered by an AC power source when the exercise mat 100 and/or accessory dashboard is plugged into an outlet.

A top surface 391 of the accessory dashboard 300 includes an engraving 379 of a wireless charger symbol. Users may charge their devices (e.g., mobile devices or smart phones) by placing the devices on or near the engraving 379. A wireless charger coil 388 and wireless charger circuit board 387 are located beneath the top surface of the accessory dashboard 300 to permit the devices to be wirelessly charged. A power indicator 380 indicates the remaining power of one or more power sources 205 (e.g., rechargeable lithium ion batteries) included within the accessory dashboard 300. One or more input devices 240 (e.g., switches 278 or buttons) permit a user to activate and deactivate accessories 150 included with the accessory dashboard 300, e.g., such as a wireless communication module (e.g., a Blue-tooth module), the wireless charger 290, and/or one or more speakers. Four sets of openings 377 are located on the top surface of the accessory dashboard 300 above speakers 383 within the accessory dashboard 300 to permit audio to be output.

A side surface 394 of the accessory dashboard 300 includes an opening that provides access to the one or more connectors 381 (e.g., USB ports). At least one of the connectors 381 can include an output that permits apparatuses (e.g., fans, mobile devices, and/or other electrically powered devices) to be connected to the accessory dashboard 300, and powered by one or more batteries 385 included in the accessory dashboard 300. At least one of the connectors 381 can include an input that allows a wire or plug to be inserted to charge the one or more batteries 385. The connectors 381 can be included on a power board 380 that is coupled to the one or more batteries 385. In some cases, the power board 380 also can include a step-up module or amplifier that increases the voltage output by the one or more batteries 385. Some or all of the accessories (e.g., such as wireless communication module 382, wireless charger circuit board 387, etc.) included within the accessory dashboard 350 also can be electrically coupled to the power board 380 to receive power from the batteries 385 connected to the power board 380.

In this example, the accessory dashboard 300 is equipped with four speakers 383 (although any number of speakers 383 may be used). In some cases, the speakers 383 are waterproof to avoid damage from the mist and/or vapor output by the aroma dispenser 210. The speakers 383 can be electrically coupled to one or more communication devices and/or interfaces 230 (e.g., a Bluetooth communication module 382). The Bluetooth communication module 382 also can include an amplifier (e.g., which can increase the amplitude of signals provided to the speakers 383). The Bluetooth communication module 382 can be paired and/or coupled to a user's device (e.g., smart phone or computer), and can permit audio to be streamed to the speakers 383 included in accessory dashboard 300.

FIG. 3J demonstrates a bottom portion 395 of the accessory dashboard 300 connected to an exercise mat 100. A plurality of openings 384 (e.g., holes and/or recesses) can be cut into the exercise mat 100 to accommodate the electronic components (e.g., the aroma dispenser 310, speakers 383, battery 385, wireless coil 388, power board 380, Bluetooth communication module 382, and/or other components). In some cases, one or more of the openings 384 can extend all the way through the top surface 120 and the bottom surface 130 of the exercise mat 100. In some cases, one or more of the openings 384 can extend downward from the top surface 100, but may not extend through the bottom surface 130. The openings 384 can be sized and shaped appropriately based on the sizes and shapes of the electronic components. The openings 384 can be situated in locations that correspond to the locations of the electronic components included on the bottom portion 395 of the accessory dashboard 300.

Inserting the electronic components in the openings 384 can reduce the thickness and size the accessory dashboard 300, which can be useful to permit transportation of the exercise mat 100, avoid interfering with usage of the exercise mat 130, and reduce the weight of the exercise mat 100. Additionally, the openings 384 can be useful for securing the electronic components in place within the accessory dashboard 300.

When the bottom portion 395 of the accessory dashboard 300 is placed directly beneath the exercise mat 100, the electronic components can be accessed via the openings 384. The top portion 390 of the accessory dashboard 300 can then be placed directly on the top surface 120 of the exercise mat 100 and connected to the bottom portion 395 (e.g., using screws 389). FIG. 3E shows the top portion 390 of the accessory dashboard 300 connected to the bottom portion 395.

FIG. 3J demonstrates how the electronic components and/or accessories 150 can be integrated into the exercise mat 100 in a manner than minimizes the thickness and size of the exercise mat 100. The sub-components of each accessory can situated adjacent to each other or side-by-side in many cases to reduce the thickness. For example, rather than placing the wireless coil 388 and wireless charger circuit board 387 on top of each other (which would increase the thickness), these components can be placed adjacent to one another and/or situated in the same horizontal plane. Similarly, rather than placing the wireless communication module 382 and speakers 383 on top of one another, these components can be placed adjacent to one another and/or situated in the same horizontal plane. Along similar lines, rather than placing the battery 385 and power board 380 on top of one another, these components can be placed adjacent to one another and/or located in the same horizontal plane. The subcomponents of any other accessory mentioned in this disclosure also can be integrated into the exercise mat 100 in a similar manner by placing the subcomponents adjacent to each other and/or in the same horizontal plane.

FIGS. 3K-3N further demonstrate the appearance of the accessory dashboard 300 when the bottom portion 395 is connected to the top portion 390. In certain embodiments, rather than cutting holes into the exercise mat 100 and integrating the accessory dashboard as described above, the accessory dashboard 300 can be standalone structure that is connected to an exercise mat 100. For example, one or more surfaces of the accessory dashboard 300 (e.g., such as side surface 399) can include a connector that is configured to attach accessory dashboard 300 to exercise mats 100, which can enable traditional exercise mats (e.g., traditional yoga mats without electronics) to be retrofitted and equipped with the functionality provided by the accessory dashboard 300. As explained above, in some cases, the connector can be a universal connector that is adjustable to enable exercise mats of varying widths and thicknesses to be connected to the accessory dashboard 300.

Many other configurations of the exercise mats 100 and accessory dashboards 300 are intended to be encompassed by this disclosure. The exemplary configurations described herein are not intended to be limiting. Moreover, it should be understood that any accessories 150 mentioned in this disclosure, which are not depicted in particular drawings can be incorporated or integrated into the exercise mats 100 (e.g., using physical, electrical, and/or data connections).

In many exemplary configurations, the exercise mats 100 at least include one or more power sources 205, and one or more aroma dispensers 210 (e.g., one or more diffusers), and one or more audio devices 215. For example, the exemplary aroma dispensers 210 may include one or more oil diffusers, such as one or more ultrasonic diffusers, one or more humidifying diffusers, one or more nebulizing diffusers, one or more atomizing diffusers, one or more evaporative diffusers, one or more heat diffusers, one or more electronic diffusers, one or more electric diffusers, and/or other types of diffusers. In certain embodiments, the one or more aroma dispensers 210 may include one or more ultrasonic diffusers, each of which includes a water reservoir 195.

In some embodiments, a power source 205 (e.g., a rechargeable battery) can be located between the upper and lower surfaces of the exercise mat 100. The exercise mat 100 also can include a plurality of diffusers and a plurality of audio devices 215 (e.g., speakers that can be wirelessly paired to, and controlled by, a computing device or electronic device) that are integrated directly into the body portion of the exercise mat 100. Electrical connections (e.g., wires, wiring, and/or circuitry) also can be located between the upper and lower surfaces of the exercise mat and hidden from a user's view. The electrical connections 180 can connect each of the accessories 150 to the power source 205, thus permitting the power source 250 to supply electrical current and/or power to the accessories 150.

In some embodiments, a portion of the oil diffusers or aroma dispensers 210 (e.g., including water reservoirs 195) may be located between the bottom surface and the top surface of the exercise mat 100. A top portion of the diffusers (e.g., which may include adjustable nozzles) can extend above the top surface and can output mist or vapor when the aroma dispensers 210 is powered on and operating. In certain embodiments, each aroma dispenser 210 may include a volume output option for controlling the volume of the mist produced by the diffuser. Each diffuser also may include a mode option that permits a user to select different operational modes (e.g., a continuous diffusion mode, an intermittent diffusion mode, etc.) for the diffuser, as well as an automatic shut off feature (e.g., which may shut off when there is no water in the reservoir and/or for other reasons mentioned in this disclosure).

In certain embodiments, the entireties of the diffusers are capable of being detached from the exercise mats 100. For example, to remove a diffuser, a user may physically remove the diffuser from a recess included on the top surface of the exercise mat and may disconnect an electrical connection (e.g., by disconnecting a plug from a port located in the recess) to the power source 205. In other embodiments, only a portion of the diffusers can be detached from the exercise mat 100. For example, in some embodiments, a water reservoir may be removable for cleaning and refilling, while other portions (e.g., a diffuser housing, nozzle, vibrating component, etc.) of the diffusers are fixed to the exercise mat and are not removable. The water reservoir may be removed via an opening included on a surface (e.g., a bottom surface and/or side surface) of the exercise mat 100.

The audio devices 215 (e.g., speakers) may be integrated into the exercise mats such that a bottom portion of the audio devices 215 is located between the top and bottom surfaces, and an upper portion of the audio devices is contiguous or flush with the top surface of the exercise mat 100. In some cases, the upper portion of the audio devices may extend above the top surfaces of the exercise mat. The audio devices 215 (as well as any other accessories 150 included on the exercise mats 100) may be detached from the exercise mat 100 in the same manner as the diffusers.

In certain embodiments, the power source 205 located between the top and bottom surfaces of the exercise mat 100 and is hidden from view. The power source 205 can be removed via an opening in a surface of the exercise mat. In some cases, this may permit a user to recharge a battery without the battery being located within, or attached to, the exercise mat 100.

One or more input devices 240 can be used to control the diffusers, audio devices 215, power source 205, and/or other accessories 150 included on the exercise mat 100. The input devices 240 can be provided on the exercise mat 100 (e.g., a top surface of the exercise mat) and/or may be included directly on the accessories 150 themselves.

In certain embodiments, an accessory dashboard 300 comprises one or more diffusers or aroma dispensers 210, one or more audio devices 215 (e.g., speakers), one or more display devices 225, one or more input devices 240, and one or more power sources 205 (e.g., rechargeable batteries). The housing of the accessory dashboard 300 may be constructed of plastic (e.g., a translucent plastic), wood, and/or other materials. The input devices 240 can be used to control the diffusers, audio devices 215, power sources 205, and/or other accessories 150 included on the accessory dashboard 300.

In some cases, an accessory dashboard 300 can be included in a recess on the top surface of the exercise mat 100. A bottom portion of the accessory dashboard 300 may be located between a top and bottom surface of the exercise mat 100. A top portion of the accessory dashboard 300 may extend above the top surface of the exercise mat 100 permitting a user to access the accessories on the accessory dashboard 300.

In some cases, an exemplary exercise mat 100 can include an accessory dashboard 300 integrated between a top and bottom surface of the exercise mat 100. Again, the accessory dashboard 300 may include one or more diffusers or aroma dispensers 205, one or more audio devices 215 (e.g., speakers), one or more display devices 225, one or more input devices 240, and one or more power sources 205 (e.g., rechargeable batteries). An opening in the top surface of the exercise mat 100 can enable a user to access accessories 150 included on the accessory dashboard 300. In some cases, separate openings included on the top surface of the exercise mat 100 may be provided for each of the accessories 150. In other cases, a single opening provides access to all of the accessories located on a top surface of the accessory dashboard 300.

An opening included on a surface (e.g., a top surface, bottom surface, and/or side surface) can permit the accessory dashboard 300 to be removed from the exercise mat 100. The opening can be a re-sealable opening that provides access to a pocket or compartment that is configured to house the accessory dashboard 300. The aroma dispensers (and/or other accessories 150) can be configured to be removed or detached from the accessory dashboard 300. In some cases, only a portion of aroma dispensers (e.g., the water reservoir) can be removed from the accessory dashboard 300.

In certain embodiments, an exemplary exercise mat 100 can includes a plurality of side extensions connected to a body portion 110 of the exercise mat 100. Each of the side extensions may include a section that protrudes outward from a side surface of the exercise mat 100. Each of the side extensions may be configured to house or include at least one accessory 150. In certain embodiments, each of the side extensions may include an aroma dispenser 210, e.g., such as a diffuser. An opening included in the side extensions may receive an electrical connection 180 (e.g. a wire or cable) from the body portion of the exercise mat 100, which can be used to connect the accessories 150 included in the side extensions to a power source 205 and/or to provide data connections to one or more of the accessories 150 (e.g., a data storage device 280, processor 275, etc.).

In certain embodiments, the configuration of the side extensions enables accessories 150 included within the side extensions 160 to be removed and/or detached from the exercise mat 100. For example, the side extensions may include a re-sealable opening that enables the accessories 150 to be removed from the side extensions when the opening is not sealed.

In certain embodiments, the side extensions are also capable of being removed form the exercise mat 100. For example, the side extensions may be connected to the body portion 110 of the exercise mat 100 using one or more releasable connectors (e.g., buttons, hook-and-loop connectors, etc.) that permit the side extensions to be detached when desired.

In certain embodiments, an exemplary exercise mat 100 can include a plurality of accessory connectors. The accessory connectors permit accessories to be swapped and/or interchanged. Thus, a user can select and customize the accessories 150 that are included on, or connected to, the exercise mat 100. Each of the accessories 150 can include the same or similar connection mechanism, thereby enabling interchangeability of the accessories in each of the accessory connectors.

In certain embodiments, each accessory connector can facilitate a physical connection, electrical connection, and/or data connection between an accessory 150 and the exercise mat 100. For example, each of the accessory connectors 165 can include a recess in a surface of the exercise mat 100 that is fabricated to tightly or snugly receive a bottom portion of an accessory 150 and/or the entirety of the accessory 150. When any of the accessories 150 are inserted into the recess, they will not become dislodged or loosened because of the fitted connection provided by the recess and the accessories 150. Each of the accessory connectors also can provide an electrical connection (e.g., a USB port, micro-USB port, auxiliary port, etc.) that enables the accessories 150 to be connected to a power source 205 included in or on the exercise mat 100. Each of the accessory connectors further can provide a data connection (e.g., a data transmission cable) between the accessories 150 and the exercise mat 100. In some cases, the electrical connection and data connection can be provided by the same connection.

The accessories 150 attached to the accessory connectors can each include physical features that are configured to tightly or snugly mate with the recess of the accessory connectors. For example, a lower portion of the accessories can be shaped (e.g., in a circular or rectangular shape) to mate with a recess of the accessory connectors that has a corresponding shape (e.g., a corresponding circular or rectangular recess). Alternatively, or additionally, the entirety of the accessory housing can be in a shaped to mate with the accessory connector recess so as to allow the accessory to be completely within the recess (and not extend above the top surface of the exercise mat). Each of the accessory connectors may also include an electrical connection (e.g., a wire or plug) that can be connected to the electrical connection in the accessory connectors. Likewise, each of the accessory connectors may include a data connector that enables the accessories 150 to be connected to the data connector of the accessory connectors. In some cases, the electrical connection and data connection on the accessories 150 can be provided by the same connection.

Because each of the accessories 150 can include the same configuration for connecting to the accessory connectors 165 of the exercise mat 100, a user attach and detach the accessories 150 as desired. Moreover, the user can continuously change and customize the accessories 150 that are attached to the exercise mats 150.

In certain embodiments, one or more of the exercise mats 100 described herein may further include a storage compartment. The storage compartment can be incorporated in the body portion 110 of the exercise mats 100. A storage compartment can additionally, or alternatively, include or bag or pouch that is connected to the exercise mat 100. The storage compartment can be detachable from the exercise mat 100 in some embodiments. Users may place items (e.g., smart phones, jewelry, towels, etc.) inside the storage compartment.

In certain embodiments, one or more of the exercise mats 100 described herein may further be configured to receive commands and/or communicate with a remote control device (e.g., a handheld device). The remote control device may include buttons, options, and/or other input devices to transmit commands to the exercise mats 100 for controlling the exercise mats 100 and/or the accessories 150 include on the exercise mats 100. The remote control device can be configured to control any of the functionalities described herein with respect to the exercise mats 100 and/or accessories 150 (e.g., for adjusting the volumes and modes of the aroma dispensers 210, changing music or audio output by the audio devices 215, changing content that is displayed or output by displayed devices 225, etc.).

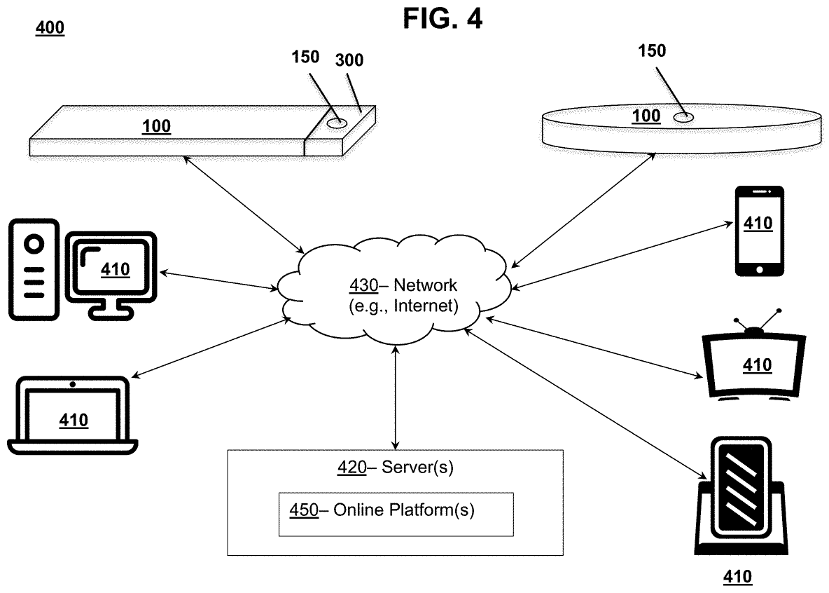

FIG. 4 is a diagram of an exemplary system 400 according to certain embodiments. The system 400 comprises one or more exercise mats 100, one or more computing devices 410, and one or more servers 420 that are in communication over a network 430. One or more of the exercise mats 100 may include an accessory dashboard 300. One or more online platforms 450 are stored on, and executed by, the one or more servers 120. The network 430 may represent any type of communication network, e.g., such as one that comprises a local area network (e.g., a Wi-Fi network), a personal area network (e.g., a Bluetooth network), a wide area network, an intranet, the Internet, a cellular network, a television network, and/or other types of networks.

All the components illustrated in FIG. 4, including the exercise mats 100, accessory dashboards 300, computing devices 410, servers 420, and online platforms 450, can be configured to communicate directly with each other and/or over the network 430 via wired or wireless communication links, or a combination of the two. Each of the exercise mats 100, accessory dashboards 300, computing devices 410, servers 420, and online platforms 450 also can be equipped with one or more communication devices and/or interfaces 230 (including any of the communication devices and/or interfaces 230 mentioned in this disclosure), one or more computer storage devices 280 (including any of the storage devices 280 mentioned in this disclosure), one or more processors 275 (including any of the processors 275 mentioned in this disclosure) that are capable of executing computer program instructions, and one or more display devices 225 (including any of the display devices 225 mentioned in this disclosure). The computer storage devices can be physical, non-transitory mediums.

In certain embodiments, the computing devices 410 can be desktop computers, laptop computers, smart phones, tablet devices, smart television smart mirrors, television peripherals, and/or other devices. In certain embodiments, the computing devices 410 can be mobile devices. A mobile electronic device can refer to a portable electronic device (e.g., an electronic device easily conveyable by hand by a person of average size) with the capability to present audio and/or visual data (e.g., text, images, videos, music, etc.). For example, a mobile electronic device can comprise at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.).

Exemplary mobile devices can comprise (i) an iPod®, iPhone®, iTouch®, iPad®, MacBook® or similar product by Apple Inc. of Cupertino, Calif., United States of America, (ii) a Blackberry® or similar product by Research in Motion (RIM) of Waterloo, Ontario, Canada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile device can comprise an electronic device configured to implement one or more of (i) the iPhone® operating system by Apple Inc. of Cupertino, Calif., United States of America, (ii) the Blackberry® operating system by Research In Motion (RIM) of Waterloo, Ontario, Canada, (iii) the Palm® operating system by Palm, Inc. of Sunnyvale, Calif., United States, (iv) the Android™ operating system developed by the Open Handset Alliance, (v) the Windows Mobile™ operating system by Microsoft Corp. of Redmond, Wash., United States of America, or (vi) the Symbian™ operating system by Nokia Corp. of Keilaniemi, Espoo, Finland.

A wearable user computer device can refer to an electronic device with the capability to present audio and/or visual data (e.g., text, images, videos, music, etc.), and that is configured to be worn by a user and/or mountable (e.g., fixed) on the user of the wearable user computer device (e.g., sometimes under or over clothing; and/or sometimes integrated with and/or as clothing and/or another accessory, such as, for example, a hat, eyeglasses, a wrist watch, shoes, etc.). In many examples, a wearable user computer device can comprise a mobile electronic device, and vice versa. However, a wearable user computer device does not necessarily comprise a mobile electronic device, and vice versa. In certain examples, a wearable user computer device can comprise a head mountable wearable user computer device (e.g., one or more head mountable displays, one or more eyeglasses, one or more contact lenses, one or more retinal displays, etc.) or a limb mountable wearable user computer device (e.g., a smart watch). In these examples, a head mountable wearable user computer device can be mountable in close proximity to one or both eyes of a user of the head mountable wearable user computer device and/or vectored in alignment with a field of view of the user.

A smart television can include a television that is able to communicate over a television network and also capable of performing one or more additional functions, such as communicating over non-television networks (e.g., the Internet, a LAN, a PAN, etc.), executing applications (e.g., native apps or applications), and/or performing other computing functions. A smart mirror may include a device that functions as a mirror, and also is capable of performing one or more additional functions (e.g., displaying multimedia, communicating over one or more networks, etc.) and/or includes one or more digital components (e.g., processors, storage devices, display devices, transceivers, etc.). Exemplary smart mirrors are currently offered by Curiouser Products, Inc. A television peripheral can include any device that is capable of communicating with a television device including, but not limited to, gaming consoles (e.g., such as a version of Xbox®, PlayStation®, etc.), set-top boxes, digital media players (e.g., Apple TV®, Amazon Fire Sticks®, etc.), and/or other similar devices.

The one or more servers 420 may generally represent any type of computing device, including any of the computing devices 410 mentioned above. In certain embodiments, the one or more servers 420 comprise one or more mainframe computing devices that execute web servers for communicating with the computing devices 410, exercise mats 100, and other devices over the network 430 (e.g., over the Internet).

Each of the one or more computing devices 410 can be configured to communicate with the exercise mats 100 and/or any of the accessories 150 included on the exercise mats 100 over a wired and/or wireless communication channel. In certain embodiments, the one or more computing devices 410 can communicate with the exercise mat 100 and/or accessories over a network 430, e.g., such as a network that includes a LAN, WAN, PAN, cellular network, television network, and/or other network. The one or more computing devices 410 can additionally, or alternatively, communicate directly with the exercise mat 110, accessory dashboard 300, and/or accessories 150. For example, in certain embodiments, the one or more computing devices 410 can be coupled directly to an exercise mat 100, accessory dashboard 300, and/or one or more accessories 150 included on the exercise mat 100 using a pairing procedure, and the one or more computing devices 410 can communicate directly with the exercise mat 100 and/or accessories 150 once the paring procedure has completed.

In certain embodiments, each computing device 410 can include and/or display one or more graphical user interfaces (GUIs). The GUIs can comprise text and/or graphics (e.g., images/videos) based user interfaces. In the same or different embodiments, the GUIs can comprise a heads up display (“HUD”).

In certain embodiments, the one or more computing devices 410 can control the functionality of some or all the accessories 150 included on the exercise mat 100. In certain embodiments, each of the computing devices 410 may store an application that is configured to control the functionality of the accessories 150 included on the exercise mat 100 and/or accessory dashboard 300. Additionally, or alternatively, the computing devices 410 may access an online platform 450 that includes an application for controlling the functionality of the accessories 150 included on the exercise mat 100 and/or accessory dashboard 300.

In certain embodiments, the exercise mats 100 and/or accessories 150 included on the exercise mats 100 each may include one or more communication interfaces or devices 230 (e.g., transceivers and/or hardwire connections) that enable the computing devices 410 to communicate with the exercise mats 100 and/or accessories. In some cases, one or more communication interfaces or devices 230 included on an exercise mat 100 may be in communication with a controller included on the exercise mat 100, and a computing device 410 may transmit commands to the controller via the one or more communication interfaces or device 230 in order to control the functionalities of the accessories 150 included on the exercise mat 100. Additionally, or alternatively, each of the accessories 150 included on an exercise mat 100 may include one or more communication interfaces or devices 230, and the computing devices 410 may transmit commands and/or other data directly to the accessories 150 to control the functionalities of the accessories 150. The computing devices 410 and/or applications included on the computing devices 410 can be configured to control any of the accessory functionalities mentioned in this disclosure.

In certain embodiments, the online platform 450 is stored on, and executed by, the one or more servers 120. The online platform 450 can be configured to perform various functions associated with enhancing functionalities of the exercise mats 100 and associated accessories, as well as enhancing experiences of users who utilize the exercise mats 100. Any functionality of the online platform 450 mentioned in this disclosure can additionally, or alternatively, be performed by an application (e.g., a mobile app) stored on a computing device 410.

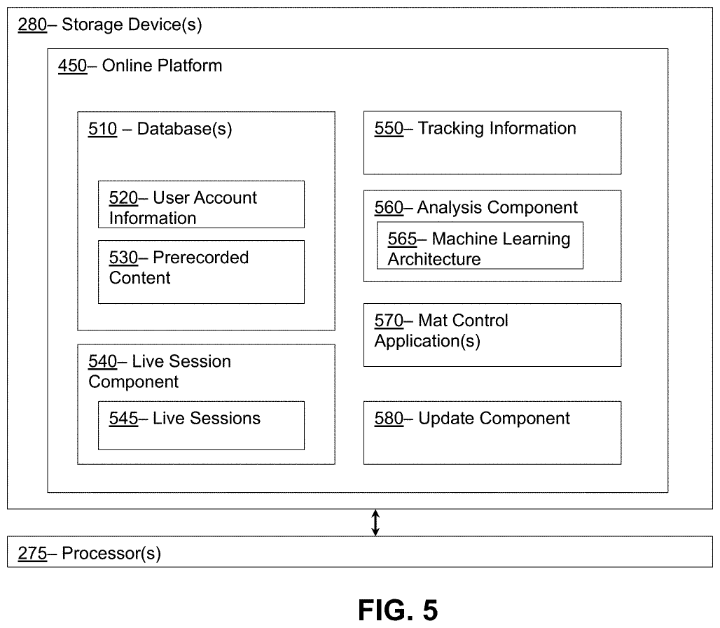

FIG. 5 is a block diagram of an exemplary online platform 450 in accordance with certain embodiments. The online platform 450 includes one or more storage devices 280 that are in communication with one or more processors 275. The one or more storage devices 280 can store data and instructions associated with one or more databases 510, user account information 520, pre-recorded content 530, a live session component 540, tracking information 550, analysis component 560, one or more mat control applications 570, and/or an update component 580. The one or more processors 275 are configured to execute instructions associated with these components and associated subcomponents. Each of these components and subcomponents are described in further detail below.

The online platform 450 may store one or more databases 510. The databases 510 can store any data and/or information associated with the online platform 450. Exemplary information stored in the database may include user account information 520, prerecorded sessions 530, multimedia content (e.g., music, videos, etc.), tracking information 550, and/or other data.

The user account information 520 may include any information associated with customer or user accounts on the online platform 450. For example, individuals who have purchased the exercise mats 100 can access the online platform (e.g., via a website or mobile application) to setup an account in order to access the functions provided by the online platform 450. Each user account may include identifying information associated with a corresponding user (e.g., name, e-mail address, phone number, username, password, etc.). Each user account may include information (e.g., product IDs, serial numbers, etc.) that identifies one or more exercise mats 100 owned by or associated with a corresponding user. Each user account also may store various information (e.g., including, but not limited, to the tracking information 550 described below) about a corresponding user's interactions with the electronic platform 450 and/or an exercise mat 100 associated with the user.

The prerecorded content 530 may relate to any type of prerecorded multimedia content. Exemplary prerecorded multimedia content may include audio and video content associated using the exercise mats 100. For example, the prerecorded content 530 may include prerecorded exercise videos (e.g., for yoga, Pilates, weight training, etc.), instructional videos, meditation music and videos, etc. Users can access the prerecorded content 530 via the online platform 450 (e.g., using a web browser or application to access the online platform 450). The prerecorded content 530 can be transmitted over a network 430 and output by accessories 150 (e.g., audio devices 215, display devices 225, etc.) included on the exercise mats 100. The prerecorded content 530 also can be transmitted over a network 430 and output by computing devices 410 operated by the users. The prerecorded content 530 also can be transmitted over a network 430 and output by televisions operated by the users.

The live session component 540 can be configured to establish live sessions 545 over the network 430 between the online platform 450 and exercise mats 100 and/or between the online platform 450 and the computing devices 410. The live sessions 545 may include real-time videoconference sessions. In certain embodiments, the live sessions 545 may allow users of the exercise mats to participate in live exercise sessions (e.g., live yoga classes, Pilates classes, weight training classes, etc.), live meditation sessions, and/or other live sessions. The lives sessions 545 may be transmitted over a network 430 and output to users on accessories 150 (e.g., audio devices 215, display devices 225, etc.) included on the exercise mats 100, computing devices 410 operated by the users, and/or televisions operated by the users.

The tracking information 550 can include any information associated with tracking usage of the exercise mats 100 and/or tracking information about users' who utilize the exercise mats 100. The tracking information 550 associated with each user and/or each exercise mat 100 may be associated with a specific user account on the online platform 550.

The tracking information 550 may include information or data associated with tracking a user's health and/or vitals, e.g., such as the user's weight, heartbeat, respiratory rate, respiratory effort, body temperature, blood pressure, oxygen saturation, and/or other related information. Some or all of this information 550 may be obtained from one or more sensors 260 incorporated into the exercise mats 100.

The tracking information 550 also may include information about a user's habits of using the exercise mats 100 or workout habits. For example, the tracking information 550 may indicate how frequently the user utilizes the mat, the types of exercises (e.g., yoga, Pilates, weigh training, etc.) and/or activities (e.g., meditation, camping, resting, etc.) the user engages in while using the exercise mat 100.

The tracking information 550 also may include any and all information about the usage of any accessories 150 that are included on the exercise mat 100. For example, the tracking information 550 may indicate which accessories 150 are included on the exercise mat 100, the frequency at which each of the accessories 150 are utilized by the user, the settings of the accessories 150 when they are utilized by the user, the functions of the accessories 150 that are utilized by the user, etc.

The tracking information 550 also may include any data and information that is generated by the accessories 150 included on the exercise mats. For example, the tracking information 550 may include audio/video/images captured by camera devices 220 and/or input devices 240 (e.g., microphones) included on the exercise mat 100.

The tracking information 550 also may include any and all information about users' interactions with the online platform 450. For example, the tracking information 550 may indicate any pre-recorded content (e.g., training videos, music, etc.) the user accessed, any live sessions the user engaged in, etc.

The analysis component 560 can be configured to analyze any data (e.g., including, but not limited to, the tracking information 550) and to generate various types of information and feedback for users. For example, the analysis component 560 can be configured to analyze the image and/or video content (e.g., captured by camera devices 220 included on the exercise mat 100, camera devices included on computing devices 110 of users, and/or other camera devices 220) to provide feedback and/or recommendations on exercises (e.g., yoga poses/stances, Pilates moves, weight training activities, etc.) and/or other movements performed by users who utilize the exercise mats 100. The feedback and/or recommendations may include suggestions for improving exercises and/or movements (e.g., for improving yoga poses/stances, Pilates moves, weight training activities, etc.). The analysis component 560 also can be configured to recommend new and/or alternative exercises for users.

In certain embodiments, the feedback and/or recommendations provided by analysis component 560 can be based on an analysis of the data generated by one or more sensors 260 included on the exercise mat 100. For example, the sensors 260 included on the exercise mats 100 may be configured to analyze movements of users, poses or stances of users, users' balance and alignment, pressure applied by the user on the exercise mat, and/or related information. This information can then be used to generate feedback and/or recommendations (e.g., for perfecting poses, stances, alignment, feet positions, etc.).

In one example, the sensors 260 included in the exercise mat 100 can determine locations of a user's feet and provide feedback for adjusting the locations of the user's feet. Additionally, or alternatively, video or images captured by camera devices 220 can be analyzed to provide feedback on a user's back posture and/or positions of user's arm and leg positions. The analysis component 560 can provide recommendations and/or feedback in other ways as well.

The mat control applications 570 can include one or more applications for controlling the functionality of the exercise mats 100 and/or accessories 150 included on the exercise mats 100. The mat control applications 570 can control some or all of the accessory functionalities mentioned in this disclosure. For example, for any audio components 215 included on an exercise mats100, a mat control application 570 can be used to control audio output to the audio components 215 and the volume of the audio. For any display devices 225 included on, or connected to, an exercise mat 100, the mat control application 570 can control any video and/or that is output by the display devices 225. For any aroma dispenser 210 included on, or connected to, an exercise mat 100, the mat control application 570 can control the mist output modes and mist output volumes. The mat control application 570 also can be used to activate and deactivate any accessory 150 included on, or connected to, an exercise mat 100.

In certain embodiments, the mat control application 570 can be stored a mobile application or local application on a computing device 410 operated by a user of an exercise mat 100. Additionally, or alternatively, the mat control application 570 can be implemented as a web-based application.

The update component 580 can be configured to generate and/or transmit updates to the exercise mats 100. For example, the update component 580 can update the software and/or firmware associated with the accessories 150 included on the exercise mats 100. These updates can be transmitted over the network 430 to the exercise mats 100.

In certain embodiments, the analysis component 560 includes a machine learning architecture 565 that is configured to generate recommendations and/or feedback for users of the exercise mats. In certain embodiments, the machine learning architecture 565 can execute computer vision functions associated with analyzing images and/or video content (e.g., images of user's stances, poses, and/or body positions) to determine whether users poses are correct and/or to provide recommendations for adjusting users poses and/or body positions. In some cases, the images analyzed by the machine learning architecture 565 can be captured by camera devices 220 included on the exercise mats 100 and/or included on computing devices 410 utilized by users.

The configuration of the machine learning architecture 565 can vary. In certain embodiments, the machine learning architecture 565 can include one or more learning models, such as one or more machine learning models, one or more artificial intelligence (Al) models, and/or one or more neural network models.

In certain embodiments, the machine learning architecture 565 may comprise a convolutional neural network (CNN), or a plurality of convolutional neural networks. Each CNN may represent an artificial neural network (e.g., that is inspired by biological processes), and may be configured to analyze images, and to execute deep learning functions and/or machine learning functions on the images. Each CNN may include a plurality of layers including, but not limited to, one or more input layers, one or more output layers, one or more convolutional layers (e.g., that include learnable filters), one or more ReLU (rectifier linear unit) layers, one or more pooling layers, one or more fully connected layers, one or more normalization layers, etc.

The configuration of the one or one CNNs and their corresponding layers enable the CNNs to learn and execute various functions for analyzing, interpreting, and understanding the images including, but not limited to functions for understanding the proper positioning of human bodies and/or anatomical parts when performing each of a plurality of poses (e.g., yoga poses). In some embodiments, the one or more CNNs may initially be trained to perform human parsing functions that are able to segment or parse an individual's body into various anatomical components (e.g., head, arms, torso, etc.). For each of a plurality of poses, the one or more CNNs can then be trained to understand propositioning of a human body (or its anatomical components) when performing the pose. After training, the machine learning architecture 365 can analyze images of exercise mat users to determine whether poses are performed correctly and/or generate analysis results providing recommendations for improving poses (e.g., adjusting position of anatomical components and/or postures). Exemplary configurations of the machine learning architecture 565 are discussed in further detail below.

In certain embodiments, the machine learning architecture 565 can be trained to perform these and other computer vision functions using any supervised, semi-supervised, and/or unsupervised training procedure. In certain embodiments, the machine learning architecture 565, or portion thereof, is trained using a supervised or semi-supervised training procedure. In certain embodiments, the machine learning architecture 565 can be trained using training images. For example, for each pose, separate training images may be provided that demonstrate proper performance of the pose (e.g., when show a human with a proper body position). In some cases, training images of incorrect poses also may be analyzed and they may include annotations that identify anatomical features that are not in the proper position. Thus, the training images can enable the machine learning architecture 565 to learn both proper and improper positioning of human bodies and/or anatomical components when performing each of a plurality of poses. In some cases, one or more loss functions may be utilized to guide the training procedure applied to the machine learning architecture 565.

In certain embodiments, the machine learning architecture 565 can use a pre-existing backbone network or model to analyze the images. Exemplary networks or models that can be used may include DeepLabV3, ResNet101, U-Net, and/or other similar neural network. In some cases, the backbone network can initially be used to predict a segmentation map for each anatomical feature (e.g., full body, torso, arm, legs, etc.) of a human depicted in an image. These backbone networks, or other neural networks, can be further adapted and/or trained to determine the accuracy of poses and/or to generate feedback for improving or correcting poses.

In certain embodiments, any or all of the functions performed by the online platform 450 can additionally, or alternatively, be executed by the exercise mats 100 and/or computing devices 410.

It should be recognized that any features and/or functionality described for an embodiment in this application can be incorporated into any other embodiment mentioned in this disclosure. The embodiments described in this disclosure can be combined in various ways. Any aspect or feature that is described for one embodiment can be incorporated into any other embodiment mentioned in this disclosure. Moreover, any of the functions performed by the exercise mats 100, accessories 150, accessory dashboards 300, online platform 450, and/or electronic devices 410 described herein may be hardware-based, may be software-based, or may comprise a mixture of both hardware and software elements. Thus, while the description herein may describe certain embodiments, features, or components as being implemented in software or hardware, it should be recognized that any embodiment, feature, or component that is described in the present application may be implemented in hardware and/or software.

In certain embodiments, an exercise mat comprises: a body portion, wherein the body portion comprises a top surface, a bottom surface, and one or more side surfaces; one or more power sources integrated into the exercise mat; and one or more electronic accessories integrated into the exercise mat; wherein the one or more electronic accessories integrated into the exercise mat are configured to be electrically connected to the one or more power sources and configured to be powered by the one or more power sources.

In certain embodiments, an exercise mat comprising: a body portion, wherein the body portion comprises a top surface, a bottom surface, and one or more side surfaces; an accessory dashboard structure coupled to an end portion of the exercise mat and including a housing; one or more power sources; and one or more electronic accessories; wherein each of the one or more power sources and each of the one or more electronic accessories are included with the housing of the accessory dashboard structure or connected to the housing of the accessing dashboard structure; and wherein each of the one or more electronic accessories are configured to be electrically connected to the one or more power sources and configured to be powered by the one or more power sources.

In certain embodiments, an exercise mat comprises: a body portion, wherein the body portion comprises a top surface, a bottom surface, and one or more side surfaces; one or more openings or recesses that extend into the top surface of the body portions; one or more power sources integrated into the exercise mat; and one or more electronic accessories integrated into the exercise mat; wherein the one or more electronic accessories integrated into the exercise mat are configured to be electrically connected to the one or more power sources and configured to be powered by the one or more power sources; wherein at least a portion of the electronic accessories are integrated into the one or more openings or one or more recesses.

In certain embodiments, a method of providing or fabricating an exercise mat includes: integrating one or more holes or recesses into a body portion of the exercise mat; integrating one or more electronic accessories into the one or more holes or recessing; and optionally attaching an accessory dashboard to the exercise mat.

In certain embodiments, a method of providing a yoga mat comprises: providing a body portion that includes a top surface, a bottom surface, and one or more side surfaces; wherein: (i) one or more power sources integrated into the exercise mat; and (ii) one or more electronic accessories integrated into the exercise mat; wherein the one or more electronic accessories integrated into the exercise mat are configured to be electrically connected to the one or more power sources and configured to be powered by the one or more power sources.

In certain embodiments, a computer program product is provided comprising a non-transitory computer-readable medium, including instructions for causing a computer to: (i) receive, over a network at an online platform, data pertaining usage to one or more electronic accessories included on an exercise mat; and (ii) transmit, over the network, data to the exercise mat and/or a computing device operated by a user of the exercise mat.

Embodiments may include a computer program product accessible from a computer-usable or computer-readable medium providing program code for use by or in connection with a computer or any instruction execution system. A computer-usable or computer-readable medium may include any apparatus that stores, communicates, propagates, or transports the program for use by or in connection with the instruction execution system, apparatus, or device. The medium can be a magnetic, optical, electronic, electromagnetic, infrared, or semiconductor system (or apparatus or device), or may be a propagation medium. The medium may include a computer-readable storage medium, such as a semiconductor, solid state memory, magnetic tape, a removable computer diskette, a random access memory (RAM), a read-only memory (ROM), a programmable read-only memory (PROM), a static random access memory (SRAM), a rigid magnetic disk, and/or an optical disk, etc.

While various novel features of the invention have been shown, described and pointed out as applied to particular embodiments thereof, it should be understood that various omissions, substitutions, and changes in the form and details of the systems and methods described and illustrated, may be made by those skilled in the art without departing from the spirit of the invention. Amongst other things, the steps in any methods may be carried out in different orders in many cases where such may be appropriate. Those skilled in the art will recognize, based on the above disclosure and an understanding of the teachings of the invention, that the particular hardware and devices that are part of the system described herein, and the general functionality provided by and incorporated therein, may vary in different embodiments of the invention. Accordingly, the description of system components are for illustrative purposes to facilitate a full and complete understanding and appreciation of the various aspects and functionality of particular embodiments of the invention as realized in system and method embodiments thereof. Those skilled in the art will appreciate that the invention can be practiced in other than the described embodiments, which are presented for purposes of illustration and not limitation. Variations, modifications, and other implementations of what is described herein may occur to those of ordinary skill in the art without departing from the spirit and scope of the present invention and its claims.

What is claimed is:

1. An exercise mat comprising:
a body portion, wherein the body portion comprises a top surface, a bottom surface, and one or more side surfaces;
one or more power sources integrated into the exercise mat; and
one or more electronic accessories integrated into the exercise mat;
wherein the one or more electronic accessories integrated into the exercise mat are configured to be electrically connected to the one or more power sources and configured to be powered by the one or more power sources.

2. The exercise mat of claim 1, wherein:
the one or more electronic accessories integrated into the exercise mat at least include one or more electronic aroma dispensers; and
the one or more electronic aroma dispensers are configured to be electrically coupled to the one or more power sources.

3. The exercise mat of claim 2, wherein the one or more electronic aroma dispensers comprise: one or more ultrasonic diffusers, one or more humidifying diffusers, one or more nebulizing diffusers, one or more atomizing diffusers, or one or more electric diffusers.

4. The exercise mat of claim 2, wherein:
the one or more electronic aroma dispensers comprises at least one ultrasonic diffuser;
the at least one ultrasonic diffuser includes a reservoir configured to store a mixture of water and oils;
a vibration component that is configured to vibrate at an ultrasonic frequency;
an output vent configured to output a mist; and
at least a portion of the at least one ultrasonic diffuser is configured to be detachable from the exercise mat.

5. The exercise mat of claim 2, wherein the one or more electronic aroma dispensers are capable of being attached and detached from the exercise mat.

6. The exercise mat of claim 1, wherein the one or more electronic accessories integrated into the exercise mat further include:
at least one audio device, wherein:
the at least one audio device includes a speaker and is configured to be wirelessly coupled to an electronic device; and
the at least one audio device is electrically coupled to the one or more power sources.

7. The exercise mat of claim 1, wherein the exercise mat further comprises an accessory dashboard that is configured to house, or facilitate connection of, the one or more electronic accessories integrated into the exercise mat.

8. The exercise mat of claim 7, wherein the accessory dashboard is configured to be detachable from the exercise mat.

9. The exercise mat of claim 6, wherein:
the accessory dashboard at least comprises an upper surface and a bottom surface;
a recess or opening is formed between the upper surface and the bottom surface of the accessory dashboard; and
the body portion of the mat extends into the recess or opening formed between the upper surface and the bottom surface of the accessory dashboard

10. The exercise mat of claim 1, wherein:
the exercise mat further comprises one or more openings or holes that extend through the top surface and the bottom surface of the body portion; and
at least a portion of the one or more electronic accessories are situated within the one or more openings or holes.

11. The exercise mat of claim 1, wherein:
the exercise mat further includes an accessory dashboard that is configured to house, or facilitate connection of, the one or more electronic accessories integrated into the exercise mat;
the one or more electronic accessories include one or more audio devices;
the one or more electronic accessories include one or more wireless charger devices;
the one or more electronic accessories include one or more wireless communication devices;
the one or more electronic accessories include one or more aroma dispensers;
the one or more power sources include at least one battery;
the at least one battery is configured to provide power to the one or more audio devices, the one or more wireless charger devices, the one or more wireless communication devices, and the one or more aroma dispensers.

12. The exercise mat of claim 1, wherein:
the one or more electronic accessories include one or more sensors integrated into exercise mat;
the one or more sensors are configured to monitor or track movements, stances, or poses of a user on the exercise mat.

13. The exercise mat of claim 13, wherein:
an online platform or computer device is configured to receive and analyze data generated by the one or more sensors; and
the online platform or computer device is configured to generate feedback or recommendations associated with monitoring or tracking the movements, stances, or poses of the user on the exercise mat.

14. The exercise mat of claim 1, wherein:
an online platform permits a user to participate in a real-time live exercise session over a network;
a camera device included on the exercise mat or a computer device operated by the user permits exchange of video and audio data during the real-time live exercise session.

15. The exercise mat of claim 1, wherein:
a camera device included on the exercise mat or a computer device operated by a user of the exercise mat is configured to capture an image of the user performing a pose on the exercise mat;
the image is analyzed by a machine learning architecture that is configured to analyze one or more poses;
the machine learning architecture is configured to generate feedback relating to the pose; and
the feedback is output or provided to the user.

16. An exercise mat comprising:
a body portion, wherein the body portion comprises a top surface, a bottom surface, and one or more side surfaces;
an accessory dashboard structure coupled to an end portion of the exercise mat and including a housing;
one or more power sources; and
one or more electronic accessories;
wherein each of the one or more power sources and each of the one or more electronic accessories are included with the housing of the accessory dashboard structure or connected to the housing of the accessing dashboard structure; and
wherein each of the one or more electronic accessories are configured to be electrically connected to the one or more power sources and configured to be powered by the one or more power sources.

17. The exercise mat of claim 16, wherein:
the one or more electronic accessories integrated into the exercise mat at least include one or more electronic aroma dispensers;
the one or more electronic aroma dispensers are configured to be electrically coupled to the one or more power sources; and
the one or more electronic aroma dispensers comprise: one or more ultrasonic diffusers, one or more humidifying diffusers, one or more nebulizing diffusers, one or more atomizing diffusers, or one or more electric diffusers.

18. The exercise mat of claim 16, wherein:
the exercise mat further comprises one or more openings or holes that extend into the top surface of the body portion; and
at least a portion of the one or more electronic accessories are situated within the one or more openings or holes.

19. An exercise mat comprising:
a body portion, wherein the body portion comprises a top surface, a bottom surface, and one or more side surfaces;
one or more openings or recesses that extend into the top surface of the body portions;
one or more power sources integrated into the exercise mat; and
one or more electronic accessories integrated into the exercise mat;
wherein the one or more electronic accessories integrated into the exercise mat are configured to be electrically connected to the one or more power sources and configured to be powered by the one or more power sources;
wherein at least a portion of the electronic accessories are integrated into the one or more openings or one or more recesses.

20. The exercise mat of claim 19, wherein:
an accessory dashboard is integrated into, or connected to, an end portion of the exercise mat;
each of the one or more electronic accessories are included within, or connected to, the accessory dashboard;
the electronic accessories at least include:
an electronic aroma dispenser;
an audio device;
a wireless communication device, the wireless communication device at least enabling audio to be wirelessly transmitted to the audio device;
a wireless charging coil;
wherein the electronic aroma dispenser, the audio device, the wireless communication device, and the wireless charging coil are configured to be connected to the one or more power sources and configured to be powered by the one or more power sources.
